(19) United States
(12) Patent Application Publication
(10) Pub. No.: US 2022/0401012 A1
(43) Pub. Date: Dec. 22, 2022

(54) WEARABLE SYSTEMS FOR MEASURING SWEAT RATE AND METHODS OF USING THE SAME

(71) Applicants: Xsensio SA, Lausanne (CH); ECOLE POLYTECHNIQUE FEDERALE DE LAUSANNE (EPFL), Lausanne (CH)

(72) Inventors: Fabien Patrick Wildhaber, Troistorrents (CH); Junrui Zhang, Chongqing, Yu Bei District (CN); Hoël Maxime Guérin, Lausanne (CH); Marc Joho, Humlikon (CH); Pietro Clement, Leggiuno (IT); Raphaël Philippe Tonione, Lausanne (CH)

(21) Appl. No.: 17/776,954

(22) PCT Filed: Nov. 20, 2020
(86) PCT No.: PCT/EP20/82969

§ 371 Date: May 13, 2022

Related U.S. Application Data
(60) Provisional application No. 62/939,308, filed on Nov. 22, 2019.

(51) Int. Cl. A61B 5/00; A61B 5/145

(52) U.S. Cl. CPC A61B 5/4266; A61B 5/14517; A61B 2562/02; A61B 2562/12

(57) ABSTRACT

Presented herein are systems and methods for measuring sweat rate of a subject using a wearable system. A sweat rate may be determined automatically based on one or more signals produced by a wetting sensor module in response to a presence of sweat in the wearable system. The one or more signals may be produced using a sweat presence monitoring device, for example comprising two or more electrodes that are operable to make conductance measurements. In some embodiments, sweat drops are periodically collected by the wearable system and individually detected by the wetting sensor module such that the sweat rate is determined based on the periodic detection of the drops. In some embodiments, an energy barrier is used to produce periodic flow of sweat through the wearable device detected by the wetting sensor module such that the sweat rate is determined based on the periodic flow.

### PRIORITY APPLICATION

This application claims the benefit of U.S. Provisional Patent Application No. 62/939,308, filed Nov. 22, 2019, the contents of which are incorporated by reference herein in their entirety.

### FIELD

This disclosure relates generally to systems and methods for collecting and analyzing biofluids. In certain embodiments, the system is or comprises a wearable device for the collection and analysis of sweat from the skin of a user, including identification of a sweat rate.

### BACKGROUND

Human sweat glands start in the hypodermis layer with the secretory coil collecting water from interstitial fluid due to osmotic pressure. Then it extends through the dermis layer to the epidermis layer, in which the sweat ducts contribute to partitioning of ions, small molecules, and metabolites into the sweat. Finally, the upper coiled duct emerges at the surface of the epidermis, bringing sweat together with the rich bio-information to the surface. However, as soon as sweat comes into contact with the skin surface, it suffers from evaporation, contamination from dead skin cells, microbes, old sweat, and the like, and degradation of biomarkers may result, due to chemical instability. Moreover, many biomarkers' concentrations in sweat are known to be closely related to sweat rate. This makes it difficult to accurately analyze sweat composition and its relevance to health.

Electrochemical sensors have been widely employed for decades, and the correlation of sweat biomarkers to health status has been investigated. In certain existing sweat collection methods, sweat is induced (chemically or thermally). For example, 10-300 μL of sweat may be collected with a collector, and sent to a lab for analysis by a dedicated machine. This process is time consuming and often no more convenient than a blood test. Furthermore, the degradation of some biomarkers starts as soon as the sweat is collected. Wearable sweat monitoring devices have been introduced to overcome this problem. However, such devices may insufficiently account for varying sweat rates, among other limitations.

Therefore, there is a need for a non-invasive system for continuous sweat sampling and analysis that limits sweat contamination and evaporation, while detecting sweat rate and accounting for sweat rate in its identification and analysis of biomarkers.

### SUMMARY

Sweat emerges as droplets on human skin from sweat ducts that are about 30 μm in diameter and that are distributed on the skin surface with a density in the order of 1 per mm². Described herein are systems for non-invasive, continuous detection of sweat and automatic determination of sweat rate. In some embodiments, the system is wearable. In certain embodiments, said sweat rate determination is used to automatically provide more accurate identification and/or analysis of biomarkers in the sweat of a subject.

In some embodiments, where sweat rate is low to moderate (referred to here for simplicity as “51” for situation 1), when a droplet is collected from a sweat duct, it then flows through a microfluidic system (e.g., from a collection module to a main sensing module) until it reaches an outlet (e.g., at or in a waste module), still as a droplet with substantially the same volume as its volume at time of collection, until the next droplet emerges from the sweat duct and is collected, and so on. Therefore, the fluid flows through the whole microfluidic system drop after drop in a periodic (e.g., pulsatile) way, each sweat duct releasing a droplet periodically into the system. In some embodiments, a wetting sensor module including one or more sweat presence monitoring devices is used to measure this periodic presence and flow of sweat droplets through the system. For example, a conductance sensor acting as a sweat presence monitoring system can output signal(s) when sweat is present, thereby allowing a conductance measurement to be made that produces the signal(s), in order to determine sweat rate.

At high sweat rate, collection structure(s) used to collect droplets at the skin surface may overflow, and the wetting sensor module used to determine sweat rate described for S1 may come into saturation. In other words, by saturating the structure(s) (e.g., microfluidic channels), i.e. having a channel completely full of fluid, a conductance signal will become constant over time, thus preventing repeated and oscillatory detection of fluid through the wetting sensor module. In certain embodiments, a system as described operating in S1 can be dimensioned so that it never saturates, for example by particular placement of sweat presence monitoring device(s), or sensing elements thereof.

In some embodiments, where sweat rate is moderate to high (referred to here for simplicity as “S2” for situation 2), where the sensing principle described for S1 may become ineffective through saturation (in some cases), the collected sweat may also accumulate and, for example, merge within a fluidic capacitance in the fluidic path (e.g. in a collection module, in a main sensing module and/or at an outlet). A fluidic capacitance is generally a dead volume that the sweat must fill before it can flow further through a system. A fluidic capacitance may comprise, for example, a cavity, a lens of fluid confined by hydrophobic forces, and/or a fluidic channel (linear channel or slit channel). Any module of a system may contribute to fluidic capacitance (e.g. a collection zone, a fluidic delivery channel to sensor(s), and/or a fluidic channel to an outlet may all, individually or in some combination, contribute). Sweat may be confined within a fluidic capacitance by presence of an energy barrier downstream (e.g., somewhere along an outlet of the fluidic capacitance). This energy barrier may comprise one or more of a hydrophobic surface (e.g., a hydrophobic step or a hydrophobic surface forming a constriction), a surface tension barrier (e.g., in the form of a gap filled with gas (e.g. air) or a fluidic immiscible with water (e.g. oil)), a sterical obstacle, and a gravitational barrier. An energy barrier may be a structure promoting fragmentation of drops off of a fluid volume (be a “drop fragmenter”). A fluidic path after an energy barrier may be designed to be energetically favorable environment for sweat that passes the energy barrier (e.g., may be hydrophilic, and/or disposed at a lower gravitational potential). When enough sweat has reached the fluidic capacitance, and if the pressure (e.g. generated by the sweat ducts, and/or generated from the surface energy forces e.g., the surface tension) is sufficient, part of the sweat will eventually overcome the energy barrier and continue along the energetically favorable fluidic path downstream. In this situation, the sweat will be transported through the energy barrier in a periodic (e.g., pulsative) way, drop by drop. The volume of such a drop depends mainly on the geometry and surface properties at the location of droplet generation. The frequency of the droplet pulse is mainly a function of sweat rate and therefore appropriate measurement of this process can be used to determine the sweat rate.

Both S1 and S2 can be experienced for a given subject (e.g., human) with a particular (e.g., wearable) system. In order to cover a higher sensing range, a device could integrate two or sweat presence monitoring devices in a wetting module sensor, at least one each adapted for S1 and adapted for S2. Furthermore, a single system may be designed in such way that can determine sweat rate using a wetting sensor module in both S1 and S2 (e.g., using a single sweat presence monitoring device). For example, if S1 conditions are experienced, it may take more time for a fluidic capacitance to fill, but eventually S2-like conditions may be achieved (e.g., after filling) and the filling time can be appropriately accounted for in determining sweat rate.

In both S1 and S2, a sweat presence monitoring device (e.g., conductance sensor) of a wetting sensor module will also give a pulsatile signal corresponding to the passing of the droplets on the sensor, each pulse corresponding to one droplet (e.g., being fragmented or flowing past). Since the droplet size can be estimated (e.g. based on the geometry and surface energy considerations), it is possible to obtain an estimate of the sweat rate on the collection zone from the analysis of the conductance change over time.

In S1, the relation linking conductance change over time to sweat rate is simpler at low sweat rate, when it is very unlikely that two sweat droplets would be collected at the same time (an arborescent collection structure as described subsequently can be used to address a plurality of pores). However, as the sweat rate increases, this situation becomes more likely to occur, and more complex modelling can link conductance and sweat rate, for example using frequency spectrum analysis (e.g. fast Fourier transform, spectrogram or FFT based time-frequency analysis) or a period measuring technique (e.g. rising/fall edge detection), for example where an energy barrier is not used to force clear periodic flow of sweat (e.g., drop fragmentation). In S2, similar models can be used, but the use of an energy barrier may simplify detection of periodic sweat flow.

A system, whether designed for S1 and/or S2 conditions, can be leveraged as fluid generation rate (e.g. sweat rate) meter, thanks to its drop-to-drop resolution. A drop of fluid travelling in a channel can be monitored through different methods: optical (e.g. fluorescence) and/or electrical (e.g. conductance). The optical and/or electrical sweat presence monitoring device(s) will output one or more signals accordingly. Generally, the optical or electrical signal(s) will change (e.g., in intensity) with the level of fluid in the channel, thereby allowing temporally resolved measurements (e.g., conductance measurements) to be performed and sweat rate determined.

In a system operating according to S1 conditions, in a use case where fluid is generated continuously at a certain generation rate, and this generation rate is of interest, the fluidic structure's drop-to-drop resolution can be leveraged as described in the two phases below, as an example.

1. Once a drop of fluid gets in contact with a collection zone of a fluidic structure, it spreads and fills in the whole channel, resulting in a certain level of fluid inside the channel. The signal intensity rises within this phase.

2. When the fluid front gets in touch with a drain material (e.g., of a waste module), due to the fact that the drain material has energetic favorability (e.g., a higher hydrophilicity than the channel), most of the fluid spread in the channel in phase 1 will be transferred to (e.g., absorbed by) the drain material. The signal intensity drops within this phase.

In some embodiments, in a system operating according to S2 conditions, where collected sweat accumulates in a fluidic capacitance, the sweat exits the fluidic capacitance and progresses across the energy barrier in a periodic (e.g., pulsative) way, consistently drop after drop even when the sweat rate increases (or lowers).

This can be leveraged as a fluid generation rate (e.g. sweat) meter, as the drops travelling across the energy barrier (or further downstream) can be monitored as described (optical or electrical method) throughout this disclosure.

In some embodiments, where fluid is generated continuously at a certain generation rate, and this generation rate is of interest, the drop-after-drop flow across the energy barrier also takes place in two cyclic phases (after the fluidic capacitance has been initially filled up with fluid):
1) When additional sweat is reaching the fluidic capacitance and the pressure is sufficient for the fluid to overcome the energy barrier, a fraction of the fluid wets the energy barrier. The signal intensity rises in this phase.
2) When the fraction of fluid wetting the energy barrier comes in contact with the energetically favorable environment downstream of the energy barrier, the fluid will fraction in two parts: the main front of the fluid on the side of the fluidic capacitance relaxes at the fluidic capacitance-energy barrier interface, and a fraction of fluid will split as a drop across the energy barrier towards the energetically favorable environment for the fluid downstream. The signal intensity drops within this phase.

With the periodic alternating of the two above described phases (regardless of S1 or S2 conditions), it is possible to analyze the fluid generation rate with signal processing methods.

For example, while not wishing to be bound to any particular theory:

A. In both S1 and S2 conditions: the frequency of this periodic alternating behavior may be proportional to the fluid generation rate. Therefore, a frequency spectrum analysis method (e.g. fast Fourier transform), or a period measuring technique (e.g. rising/fall edge detection) can be used to extract the fluid generation rate and therefore sweat rate.

B. In both S1 and S2 conditions: in case that a single drop of fluid is generated continuously, if the fluidic properties are constant, the volume of drop that is spread in the channel in each period (e.g., in S1) or the volume of the drop that passes the energy barrier (e.g., in S2) will also be constant, and so is the time that the signal intensity stays high as well as the time that the signal intensity stays low. This will result in a pulse-width-modulated signal with constant duty cycle, and constant time for the high or low sections of each period, thereby allowing sweat rate to be determined.

C. In S1 conditions: it is possible that there may be multiple sources of fluid generator in contact with the same fluidic structure, but not synchronized when generating fluid flow. If our interest is the total or average fluid generation rate, the properties in example B can still be leveraged, e.g., when the fluidic properties are constant, the duty cycle will still be constant.

D. In both S1 and S2 conditions: since the intensity of a signal from a sweat presence monitoring device will be proportional to the level of fluid (e.g., inside a fluidic channel), an integration of the signal over time (tₜ) will be proportional to the total volume (Vₜ) of fluid that travelled through the channel during tₜ. Therefore, in certain embodiments, the fluid generation rate can be calculated as Vₜ/tₜ.

In certain embodiments, a system comprises (i) a collection and delivery module, (ii) a wetting sensor module, and, optionally but preferably, (iii) a waste module and/or (iv) a flow regulation module. In certain embodiments, one or more of the modules are integrated within one microchip or an assembly of microchips, for example a silicon CMOS chip, a glass chip, a printed circuit board (PCB) (e.g., a flexible PCB). For example, the modules may be mounted on a printed circuit board (e.g., a flexible printed circuit board) and/or embedded within an electronic device (e.g., in an adhesive patch or in a wearable device (e.g., a wrist-band, a head-band, a bandage, a sock, a glove, an arm-band, a waist-band, an ankle-band, and a knee-band).

In certain embodiments, the wetting sensor module includes one or more conductance sensors (e.g., one or more metal, (e.g. a noble metal, e.g. platinum or Ag/AgCl), conductive ink, or conductive polymer electrodes for measuring conductance). In certain embodiments, the conductance sensor includes one or more electrodes. In certain embodiments, the one or more electrodes includes a noble metal. In certain embodiments, the one or more electrodes are actuated by an AC signal in a frequency range of about 1 kHz to about 100 kHz. In certain embodiments, the one or more electrodes are actuated by a DC signal.

In certain embodiments, the collection and delivery module includes a collection surface, one or more collection structures, and/or one or more inlets (e.g., and a sealant material, e.g., and a spacer layer, e.g., and a filter) (e.g., for collecting a volume of the fluid from a collection zone) [e.g., for collecting a volume of the fluid from a collection zone (e.g., a region of a skin surface)].

In certain embodiments, the collection structures include at least one fluidic channel or a fluidic channel network (e.g., an arborescent channel network). A collection structure may include a cavity in which fluid (e.g., sweat) is collected, for example in combination with or as an alternative to a fluidic channel or fluidic channel network.

In certain embodiments, the at least one fluidic channel or fluidic channel network includes a member selected from the group consisting of a groove, an open or closed microfluidic channel, a two-dimensional channel defined by surface property contrast, and a channel made of a fixed gel matrix permeable to a fluid.

In certain embodiments, a portion of the at least one fluidic channel or fluidic channel network includes pillar structures (e.g., and/or pavement structures) (e.g., or arrays thereof) to reduce dead volume and/or facilitate fluid transport via capillary action (e.g., and/or to filter the fluid).

In certain embodiments, the waste module includes a capillary pump (e.g., an array of pillars (e.g. hexagonal pillars) or pavements and/or a wicking material based for instance on a paper, a textile, a gel or an absorbent material] and a waste reservoir (e.g., an absorbent pad for collecting waste) (e.g., wherein each pillar of the array of pillars has a width of between 1 μm to 1 mm and is separated from neighboring pillars by a distance of 1 μm to 1 mm) (e.g., wherein the array of pillars is a regular array, e.g., wherein the array of pillars is an irregular array).

Thus, in some aspects, the disclosure is directed to a system for automatically detecting a sweat rate of a subject, the system including: a sweat collection and delivery module; a wetting sensor module; a processor; and a memory having instructions thereon, the instructions, when executed by the processor, causing the processor to determine a sweat rate from one or more signals produced by the wetting sensor module in response to a presence of sweat from the sweat collection and delivery module.

In certain embodiments, the one or more signals produced by the wetting sensor module includes an electrical signal. In certain embodiments, the electrical signal is a conductance (e.g., a conductance detected over time) (e.g., wherein the wetting sensor module includes a conductance sensor, e.g., wherein the conductance sensor includes one or more electrodes, e.g., wherein the one or more electrodes are actuated by an AC signal in a frequency range from about 1 kHz to about 100 kHz or wherein the one or more electrodes are actuated by a DC signal). In certain embodiments, the one or more signals includes a plurality of channel conductance signals, and wherein the instructions cause the processor to determine the sweat rate from a detected frequency of conductance variation, based on the plurality of channel conductance signals. In certain embodiments, the one or more signals includes a plurality of channel conductance signals, and wherein the instructions cause the processor to determine the sweat rate from a detected duty cycle, based on the plurality of channel conductance signals.

In certain embodiments, the signal produced by the wetting sensor module includes an optical signal (e.g., a fluorescence or other optical signal detected over time).

In certain embodiments, the system includes a functionalized sensor, wherein the instructions, when executed by the processor, determine a chemical and/or physical property of the sweat (e.g., a presence of, and/or concentration of, a biomarker) based on a signal from the functionalized sensor taking into account the detected sweat rate.

In certain embodiments, the system further includes a waste module for collecting and disposing of sweat from the system after analysis. In certain embodiments, the waste module includes a capillary pump and a waste reservoir. In certain embodiments, the waste module includes a wicking material and a waste reservoir. In certain embodiments, the waste reservoir includes an absorbent pad. In certain embodiments, the waste module has an interface for vapor exchange with the atmosphere.

In certain embodiments, the system further includes a flow regulation module for regulating flow of sweat into and/or through, and/or out of the system.

In certain embodiments, the system includes a wearable housing (e.g., said housing non-invasively attachable and detachable from skin of the subject, e.g., via an adhesive surface). In certain embodiments, the wearable housing houses one or more members selected from the group consisting of the sweat collection and delivery module, the wetting sensor module, a power source (e.g., a battery), and the processor (e.g., and, optionally, the waste module and/or the flow regulation module). In certain embodiments, the processor is external to the wearable housing.

In certain embodiments, the system includes a microchip assembly for integrating at least two components selected from the group consisting of the collection and delivery module, the waste module, the flow regulation module, the processor, and the memory (e.g., wherein the microchip assembly includes a printed circuit board).

In certain embodiments, the collection and delivery module includes a surface with one or more collection structures. In certain embodiments, the collection structures include at least one fluidic channel or a fluidic channel network. In certain embodiments, the at least one fluidic channel or fluidic channel network includes one or more members selected from the group consisting of a groove, an open or closed microfluidic channel, a two-dimensional channel defined by surface property contrast, and a channel made of a fixed gel matrix permeable to a fluid. In certain embodiments, at least a portion of the at least one fluidic channel or fluidic channel network includes pillar structures to facilitate fluid transport via capillary action. In certain embodiments, the one or more collection structures include an arborescent channel network, and wherein the arborescent channel network includes a plurality of branched channels. In certain embodiments, a collection and delivery module includes collection structure that may include, for example, a cavity in which fluid (e.g., sweat) is collected, a fluidic channel or a fluidic channel network. A cavity may be used in combination with or as an alternative to a fluidic channel or fluidic channel network.

In some aspects, the disclosure is directed to a method for (e.g., automatically) detecting a sweat rate of a subject, the method including: determining, by a processor of a computing device, a sweat rate from one or more signals produced by a wetting sensor module of a wearable (e.g., by a human subject) sweat rate detection system, the one or more signals produced in response to a presence of sweat droplets in a sweat collection and delivery module of the system. In certain embodiments, the sweat rate detection system includes any one of the systems described herein.

In some aspects, the disclosure is directed to a wearable system for determining a sweat rate of a subject. The system may comprise (i) a sweat presence monitoring device (e.g., conductance sensor) comprising a first sensing element and a second sensing element (e.g., first and second electrodes) and (ii) an energy barrier. In some embodiments, the first sensing element is disposed before the energy barrier along a fluidic path of the wearable system and the second sensing element is disposed at or after the energy barrier along the fluidic path.

The energy barrier may be disposed at an outlet of a fluidic capacitance. The fluidic capacitance may further comprise one or more of a fluid collection and delivery module (e.g., portion thereof), a closed fluidic channel, and a fluid via.

In some embodiments, the energy barrier comprises one or more of a hydrophobic surface (e.g., a hydrophobic surface comprising a constriction or a hydrophobic step), a surface tension barrier, a sterical obstacle, and a gravitational barrier. In some embodiments, the energy barrier is a drop fragmenter formed from a constriction in a hydrophobic surface.

In some embodiments, the second sensing element is disposed on or in the energy barrier. In some embodiments, the energy barrier is formed from a constriction in a hydrophobic surface and the second sensing element is disposed in the constriction.

In some embodiments, the second sensing element is a ring electrode disposed (i) on or (ii) adjacent to and at the energy barrier (e.g., and forming a circular or polygonal ring). In some embodiments, the energy barrier is a ring (e.g., a circular or polygonal ring).

In some embodiments, the second sensing element is disposed in a closed fluidic channel. In some embodiments, the energy barrier is disposed in a (e.g., the) closed fluidic channel.

In some embodiments, the energy barrier and the second sensing element are disposed after a fluid via along the fluidic path. In some embodiments, one or more of the energy barrier and the second sensing element are disposed at an outlet of the fluid via (e.g., at least partially surround the outlet).

In some embodiments, the system comprises a sweat collection and delivery module comprising an inlet disposed at a beginning of the fluidic path. In some embodiments, the sweat collection and delivery module comprises a fluidic collection zone comprising an opening at the inlet (e.g., a fluidic capacitance) (e.g., comprising a shaped hydrophobic surface disposed on a hydrophilic surface). In some embodiments, a wetting sensor module comprising the sweat presence monitoring device and the sweat collection and delivery module (e.g., and the main sensor module) are integrated and disposed on or in a common (e.g., flexible) substrate. In some embodiments, a collection and delivery module includes a collection structure that may include, for example, one or more of a fluid collection zone (e.g., defined by hydrophobic and hydrophilic surfaces), a cavity in which fluid (e.g., sweat) is collected, a fluidic channel or a fluidic channel network. A cavity may be used in combination with or as an alternative to a fluidic channel or fluidic channel network.

In some embodiments, the system comprises a waste module (e.g., a capillary pump or hydrophilic wicking material) disposed along the fluidic path after (e.g., and adjacent to) the second sensing element (e.g., disposed at least partially in a closed fluidic channel).

In some embodiments, the system comprises a second sweat presence monitoring device comprising the first sensing element and a third sensing element, wherein the third sensing element is disposed after the second sensing element along the fluidic path. In some embodiments, the sweat presence monitoring device and the second sweat presence monitoring device are together operable to output temporally phase shifted signals as sweat flows along the fluidic path.

In some embodiments, the first sensing element and the second sensing element are optical or electrical sensing elements. In some embodiments, the first sensing element and the second sensing element are electrodes that are disposed such that the sweat presence monitoring device is operable to output one or more signals when sweat is disposed continuously along the fluidic path from the first sensing element to the second sensing element.

In some embodiments, the system comprises a main sensor module disposed before the first sensing element along the fluidic path.

In some embodiments, the system comprises a wearable housing that houses the fluid collection and delivery module and a wetting sensor module comprising the sweat presence monitoring device, wherein the wearable housing is non-invasively attachable and detachable from skin of the subject. In some embodiments, the wearable housing comprises a skin adhesive, wherein the sweat collection and delivery module is disposed in fluid communication with skin of the subject when the skin adhesive is adhered to the skin.

In some embodiments, the system comprises a processor; and a memory having instructions thereon, wherein the instructions, when executed by the processor, cause the processor to determine a sweat rate from one or more signals produced by the sweat presence monitoring device in response to a presence of sweat. In some embodiments, a wetting sensor module comprises the sweat presence monitoring device and further comprises a second sweat presence monitoring device comprising the first sensing element and a third sensing element, the third sensing element being disposed at or after the energy barrier along the fluidic path, and the instructions, when executed by the processor, cause the processor to determine the sweat rate based on a temporal phase shift between ones of the one or more signals and one or more signals produced by the second sweat presence monitoring device in response to a presence of sweat. In some embodiments, the instructions, when executed by the processor, cause the processor to determine the sweat rate based on a pulse frequency (e.g., duty cycle) of the signals. In some embodiments, the pulse frequency is due to individual discrete drops of sweat flowing along the fluidic path.

In some embodiments, the energy barrier is comprised in a flow regulation module.

In some aspects, the disclosure is directed to a method of determining a sweat rate of a subject. The method may comprise collecting sweat from skin of the subject in a wearable system comprising one or more sweat presence monitoring devices (e.g., conductance sensor(s)). The method may further comprises flowing the sweat over the one or more sweat presence monitoring devices over a period of time. The method may further comprise detecting that the sweat is present with the one or more sweat presence monitoring devices thereby causing the one or more sweat presence monitoring devices to output one or more signals during the period of time. The method may further comprise automatically determining, by a processor (e.g., integrated in the wearable system), a sweat rate based on a change in the one or more signals over the period of time.

In some embodiments, flowing the sweat comprises periodically flowing (e.g., at regular periods) portions of the sweat past (e.g., over or through) an energy barrier and detecting that the sweat is present occurs only as each of the portions flows past the energy barrier (e.g., wherein the energy barrier comprises one or more of a hydrophobic surface (e.g., a hydrophobic surface comprising a constriction or a hydrophobic step), a surface tension barrier, a sterical obstacle, and a gravitational barrier). In some embodiments, flowing the sweat comprises fragmenting discrete drops from the sweat by flowing the sweat past (e.g., over or through) a drop fragmenter and detecting that the sweat is present occurs only as each of the discrete drops is fragmented. In some embodiments, wherein the sweat comprises discrete drops of sweat and flowing the sweat comprises individually flowing the discrete drops over the one or more sweat presence monitoring devices. In some embodiments, flowing the sweat comprises fragmenting discrete drops from the sweat by flowing the sweat past (e.g., over or through) a drop fragmenter and detecting that the sweat is present occurs only as each of the discrete drops is fragmented.

In some embodiments, the method comprises detecting that the sweat is present with the one or more sweat presence monitoring devices after a portion of the sweat has exited a fluid via. In some embodiments, a portion of at least one of the one or more sweat presence monitoring devices (e.g., an electrode or optical sensor of the sweat presence monitoring device) (e.g., and the energy barrier) (e.g., and the drop fragmenter) is (e.g., are) disposed after the fluid via along a fluidic path of the sweat through the wearable system (e.g., and at least partially around the fluid via).

In some embodiments, collecting the sweat from the skin comprises collecting the sweat in a fluidic collection zone (e.g., comprising a shaped hydrophobic surface disposed on a hydrophilic surface). In some embodiments, collecting the sweat includes collecting in a collection structure that may include, for example, one or more of a fluid collection zone (e.g., defined by hydrophobic and hydrophilic surfaces), a cavity in which fluid (e.g., sweat) is collected, a fluidic channel or a fluidic channel network. A cavity may be used in combination with or as an alternative to a fluidic channel or fluidic channel network.

In some embodiments, each of the one or more sweat presence monitoring devices comprises a first electrode and a second electrode and detecting that the sweat is present comprises measuring a conductance through the sweat from the first electrode to the second electrode (e.g., and wherein the second electrode is disposed at or after an energy barrier) (e.g., and wherein the second electrode is disposed at or after a drop fragmenter).

In some embodiments, each of the one or more sweat presence monitoring devices is an optical sweat presence monitoring device comprising one or more optical sensing elements (e.g., that sense(s) fluorescence).

In some embodiments, the change is a change in intensity [e.g., determined using frequency spectrum analysis (e.g. fast Fourier transform, spectrogram or FFT based time-frequency analysis) or a period measuring technique (e.g. rising/fall edge detection)]. In some embodiments, the one or more signals is a plurality of signals from a plurality of sweat presence monitoring devices and the change is a temporal phase shift between ones of the plurality of signals from different ones of the plurality of sweat presence monitoring devices. In some embodiments, the one or more signals are distributed over time to form a pulse-width-modulated composite signal (e.g., with a constant duty cycle) and (e.g., automatically) determining the sweat rate is based on the pulse-width-modulated composite signal. In some embodiments, the sweat rate is a total or average fluid generation rate.

In some embodiments, the wearable system comprises the processor and a memory having instructions stored thereon, outputting the one or more signals comprises receiving the one or more signals in the processor, and the sweat rate is (e.g., automatically) determined by the processor (i) during or after the period of time upon receiving at least one of the one or more signals and (ii) using the instructions.

In some embodiments, flowing the sweat over the one or more sweat presence monitoring devices comprises passively flowing the sweat (e.g., due to one or more of fluidic (e.g., hydraulic) capacitance, the inflow of sweat with sufficient pressure generated by the sweat ducts, and capillary action). In some embodiments, flowing the sweat over the one or more sweat presence monitoring devices comprises actively flowing the sweat (e.g., by a powered pump).

In some aspects, the disclosure is directed to a wearable system for determining a sweat rate of a subject. The system may comprise a wetting sensor module comprising two sweat presence monitoring devices each comprising sensing elements (e.g., electrodes or optical sensing elements). In some embodiments, the two sweat presence monitoring devices are disposed along a fluidic path of the wearable system such that the two sweat presence monitoring devices are operable to output temporally phase shifted signals (e.g., electrical or optical signals) corresponding to sweat flow along the fluidic path. In some embodiments, the two sweat presence monitoring devices comprise a common sensing element. In some embodiments, the system comprises a fluid collection and delivery module, wherein an inlet of the fluid collection and delivery module (e.g., a fluid containment zone) is disposed at a beginning of the fluidic path.

In some embodiments, the system comprises a processor and a memory having instructions thereon, wherein the instructions, when executed by the processor, cause the processor to (e.g., automatically) determine a sweat rate from the temporally phase shifted signals. In some embodiments, an energy barrier may be disposed at an outlet of a fluidic capacitance with at least one sensing element of at least one of the two sweat presence monitoring devices disposed after the energy barrier. The fluidic capacitance may further comprise one or more of a fluid collection and delivery module (e.g., portion thereof), a closed fluidic channel, and a fluid via. In some embodiments, the system is one disclosed herein.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a block diagram of a system for collection and analysis of a fluid (e.g., a biofluid, e.g., sweat) from a surface (e.g., skin of a user), according to illustrative embodiments.

FIG. 2 is a schematic diagram showing a top view of a system for collection and analysis of a fluid from a surface, according to illustrative embodiments.

FIG. 3 is a schematic diagram showing a side view of a system for collection and analysis of a fluid from a surface, according to illustrative embodiments.

FIG. 4 is a schematic diagram showing cross sections of fluidic channels, according to illustrative embodiments.

FIG. 5 is a schematic diagram showing a system for collection and analysis of a fluid from a surface with pillar-based collection structures, according to illustrative embodiments.

FIG. 6 is a schematic diagram showing details of pillar-based collection structures, according to illustrative embodiments.

FIG. 7 is a schematic diagram showing pillar-based collection structures with an interstitial distance gradient, according to illustrative embodiments.

FIG. 8 is a schematic diagram depicting a system for collection and analysis of a fluid from a surface with arborescent collection structures, according to illustrative embodiments.

FIG. 9 is a schematic diagram showing a configuration of arborescent collection structures, according to illustrative embodiments.

FIG. 10 is a schematic diagram showing arborescent collection structures with constant angle, according to illustrative embodiments.

FIG. 11 is a schematic diagram representing the collection of a droplet of a fluid by an arborescent fluidic network in the presence of a spacer layer, according to illustrative embodiments.

FIG. 12 is a schematic diagram representing a system for sweat collection with collection structures in direct contact with the skin and sweat transport taking place within the v-groove network of the skin, according to illustrative embodiments.

FIG. 13 is a schematic diagram showing hybrid collection structures, according to illustrative embodiments.

FIG. 14 is an illustration of a main sensor module, according to illustrative embodiments.

FIG. 15 is a schematic diagram depicting a flow regulation process with a flow regulation module, according to illustrative embodiments.

FIG. 16 is a schematic diagram showing a top view of a system for collection and analysis of a fluid from a surface with a flow regulation module, according to illustrative embodiments.

FIG. 17 is a schematic diagram of a wetting sensor module, according to illustrative embodiments.

FIG. 18 is a schematic diagram of a chemical sensor activation module, according to illustrative embodiments.

FIG. 19 is a schematic diagram of a system for collection and analysis of a fluid from a surface, according to illustrative embodiments.

FIG. 20 is a schematic diagram of a system for collection and analysis of a fluid from a surface, according to illustrative embodiments.

FIG. 21 is a schematic diagram of a system for collection and analysis of a fluid from a surface, according to illustrative embodiments.

FIG. 22 is a schematic diagram of a system for collection and analysis of a fluid from a surface, according to illustrative embodiments.

FIG. 23 is a schematic diagram showing integration of a system for collection and analysis of a fluid from a surface within a flexible PCB and a patch for measurements on skin, according to illustrative embodiments.

FIG. 24 is a block diagram of an example network environment for use in the methods and systems described herein, according to illustrative embodiments.

FIG. 25 is a block diagram of an example computing device and an example mobile computing device, for use in illustrative embodiments.

FIG. 26 is a schematic diagram representing a cross-sectional view of a system to homogenously dispense fluid on the surface of a porous membrane (e.g., to mimic sweating on skin), according to illustrative embodiments.

FIG. 27 is a schematic diagram showing a top view (left) and a cross-section view (right) of a system for collection of a fluid (e.g., a biofluid, e.g., sweat) from a surface (e.g., skin of a user) by a hydrophilic channel network, according to illustrative embodiments.

FIG. 28 is a schematic diagram showing a top view (left) and a cross-section view (right) of collected droplet travelling along a sensor delivery channel, according to illustrative embodiments.

FIG. 29 is a schematic diagram showing the conductance readout as a function of time and corresponding sweat rate in a single pore scenario, according to illustrative embodiments.

FIG. 30 is a schematic diagram showing the conductance readout as a function of time and corresponding sweat rate in a multiple pores scenario, according to illustrative embodiments.

FIG. 31 is a schematic diagram the pulsative drop generation at the outlet of a fluidic capacitance across an energy barrier, according to illustrative embodiments.

FIG. 32A is a top view of and 32B is a cross-section view of a wearable system comprising a fluidic collection zone formed by a hydrophobic confinement ring acting as a fluidic capacitance, with a drop fragmenter structure as an energy barrier at the outlet, according to illustrative embodiments.

FIGS. 32C and 32D are successive views and corresponding conductance vs. time plots of sweat collection using the wearable system of FIGS. 32A-32B, according to illustrative embodiments.

FIG. 33 is cross-section view of a wearable system comprising a fluidic collection zone connected to a sensor module encapsulated in a closed fluidic channel, and a hydrophobic step as an energy barrier at an outlet, according to illustrative embodiments.

FIG. 34A is a cross-section view of and FIG. 34B is a top view of a wearable system comprising a fluidic collection zone connected to a sensor module encapsulated in a closed fluidic channel, and a drop fragmenter as an energy barrier at the outlet, according to illustrative embodiments.

FIG. 35A is a cross-section view of and FIG. 35B is a top view of a wearable system comprising a fluidic collection zone connected to a sensor module encapsulated in a closed fluidic channel, a hydrophobic ring as an energy barrier at the outlet and a circular-shaped electrode for conductance sensing, according to illustrative embodiments.

FIGS. 36A and 36B are schematic diagrams of the measurement of fragmented drop travel speed from the phase shift between two sweat presence monitoring devices that are conductance sensors, sensing elements of which are in an energy barrier, with a schematic view of the signal from both conductance sensors, according to illustrative embodiments.

Features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF CERTAIN EMBODIMENTS

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present disclosure that consist essentially of, or consist of, the recited components, and that there are processes and methods according to the present disclosure that consist essentially of, or consist of, the recited processing steps.

It should be understood that the order of steps or order for performing certain action is immaterial so long as operability is maintained. Moreover, two or more steps or actions may be conducted simultaneously.

Elements of embodiments described with respect to a given aspect of the disclosure may be used in various embodiments of another aspect of the disclosure. For example, it is contemplated that features of dependent claims depending from one independent claim can be used in apparatus, articles, systems, and/or methods of any of the other independent claims.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader. The presence and/or placement of a header is not intended to limit the scope of the subject matter described herein.

As used herein, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) where ranges are provided, endpoints are included.

As used herein, the terms “about” or “approximately”, when used herein in reference to a value, refers to a value that is similar, in context to a referenced value. In general, those skilled in the art, familiar with the context, will appreciate the relevant degree of variance encompassed by “about” or “approximately” in that context. For example, in some embodiments, the terms “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “continuous,” as in a continuous biomarker measurement, refers to performing a series of measurements (e.g., of the presence and/or quantity of a biomarker) without a substantial time interval between each measurement. For example, continuous measurements may be performed at a rate of one measurement every ten minutes, one measurement every five minutes, one measurement per minute, one measurement every 30 seconds, one measurement every 5 seconds, or faster rates.

In certain embodiments, a continuous measurement can occur in substantially “real-time” such that the concentration value of an analyte measured by the device is the concentration present in sweat without a substantial delay or latency on the timescale of physiological processes (e.g., on a scale of five minute or greater). For example, the device may display a “snapshot” of the concentration of an analyte in the biofluid (e.g., every 5 minutes, 1 minute, 30 seconds or less). In certain embodiments, the continuous measurements are performed at a higher frequency (e.g., every second or every several milliseconds) providing a continuous analyte data stream faster than the physiological timescale.

As used herein, the term “substantially” refers to the qualitative condition of exhibiting total or near-total extent or degree of a characteristic or property. For example, a substantially constant value may vary in time by 20%, 15%, 10%, 9%, 8%, 7%, 6%, 5%, 4%, 3%, 2%, 1%, or less of the constant value.

As used herein, the terms “drop” and “droplet” are used interchangeably, e.g., without particular regard to any size cutoff.

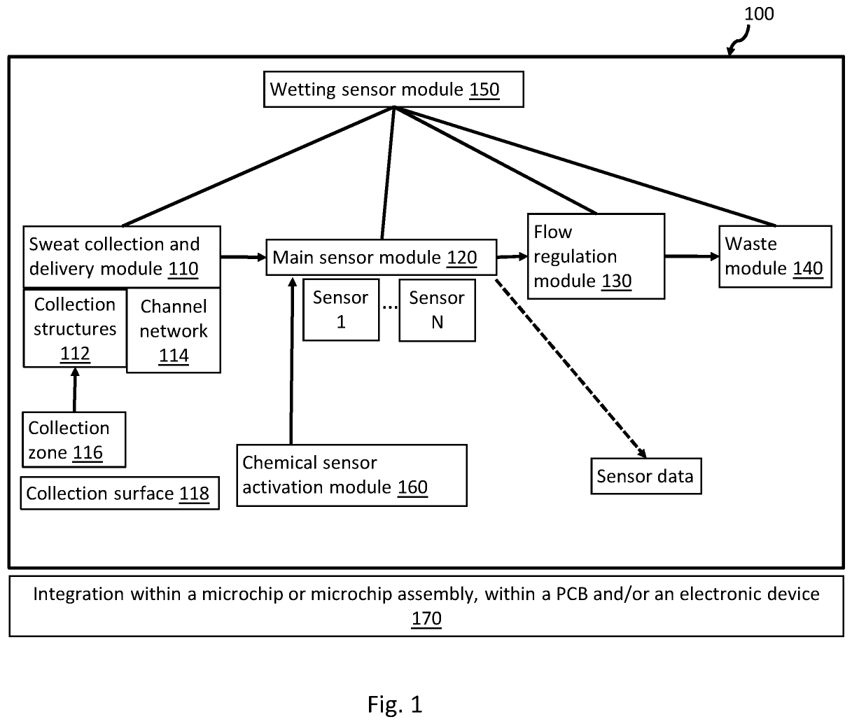

Presented herein are systems, methods for collecting fluid from a surface (e.g., skin) and analyzing the fluid (e.g., to measure chemical, physical and/or biological properties of the fluid). In certain embodiments, as shown in FIG. 1, a system 100 for fluid collection on a surface (e.g., skin) and fluid analysis includes at least one of the following modules: a collection and delivery module 110 to collect a fluid over a wet or partially wet surface and deliver it to a main sensor module 120 to perform chemical, physical and/or biological analysis on the fluid. The system also includes a flow regulation module 130, for controlling fluid flow (e.g., transport) through the system and a waste module 140 to collect and/or dispose of the fluid after analysis is complete.

In certain embodiments, system 100 includes a wetting sensor module 150 based on conductance measurements, which are used to determine whether some or all of the above-mentioned modules are wet (e.g., exposed to a fluid) and to provide an estimate of the flow rate as the system is filled. In certain embodiments, the system includes a module 160 to chemically activate a sensor (e.g., a single time or repeatedly) using a dedicated fluid delivery system. In certain embodiments, each of modules 110, 120, 130 and 140 and optional modules 150 and 160 are integrated within one microchip or an assembly of microchips. System 100 can be disposed on a substrate 220 which can provides mechanic support and electrical connections to system 100. In certain embodiments, substrate 220 includes a printed circuit board, a polymer substrate (e.g., plastic), or a metal substrate. For example, the modules may be mounted on a printed circuit board (e.g., a flexible printed circuit board) and/or embedded within an electronic device (e.g., in an adhesive patch or in a wearable device (e.g., a wrist-band, a head-band, a bandage, a sock, a glove, an arm-band, a waist-band, an ankle-band, and a knee-band).

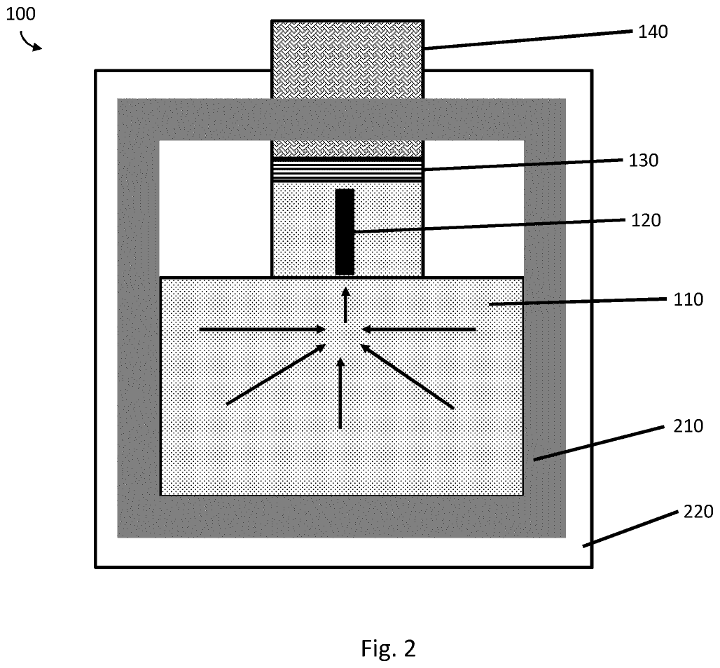

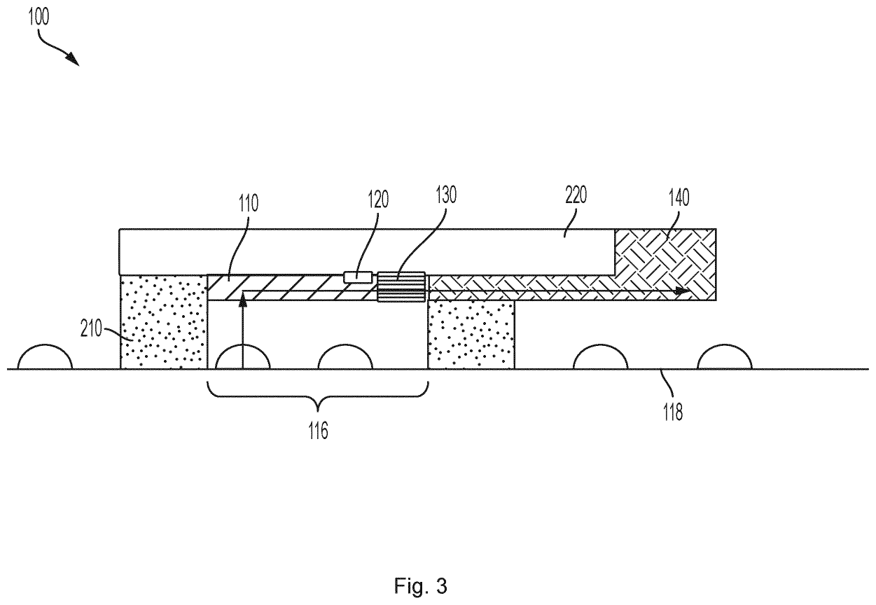

Details regarding various embodiments of a system for collection and analysis of a fluid (e.g., a biofluid, e.g., sweat) from a surface (e.g., skin of a user) are provided herein. FIG. 1 shows a schematic diagram of system 100, according to illustrative embodiments. FIG. 2 and FIG. 3 each show a schematic diagram of a top-down view and side view of the system, according to various embodiments. Modules 110, 120, 130, 140, 150 and 160 of FIG. 1, FIG. 2, and FIG. 3 are described below.

### Collection and Delivery Module

A surface (e.g., skin) can become wet (e.g., or semi-wet) by fluid from, for example, condensation, diffusion, permeation through pores and/or ducts in a person's skin. The fluid may be a biofluid, such as sweat emerging from sweat ducts on the surface of a person's skin.

As shown in FIGS. 1-3, in certain embodiments, collection and delivery module 110 includes a surface patterned with structures 112 (e.g., collection structures) that are designed to collect fluid (e.g., a biofluid, e.g. sweat) on another wet (e.g., semi-wet) surface 118 (e.g., skin) that is in contact with (e.g., substantially in contact with) surface with the collection structures 112. Fluid is collected in a collection zone 116 of the collection surface 118 (e.g., a region of skin addressed by the surface of the device) and directed through one or more fluidic channels 114 (e.g., a fluidic channel network) towards main sensor module 120. Collection proceeds in a manner that optimizes the delay between the emergence of the fluid on the collection zone and the readout by the sensors. In certain embodiments, fluid flow is driven passively (e.g., by capillary action). In other embodiments, active flow is used (e.g., driven by one or more fluid pumps, e.g., pumps requiring power).

To efficiently deliver fluid from collection structures 112 of collection and delivery module 110 to main sensor module 120, a channel network 114 is used, in certain embodiments, and is designed to direct (e.g., funnel) fluid from collection structures 112 to a sensor in main sensor module 120. In certain embodiments, channel network 114 is designed to direct the fluid in an unidirectional flow (e.g., to improve the response time of the sensor). In certain embodiments, channel network 114 is designed to ensure that the entire surface of a sensor is in contact with the collected fluid (e.g., to improve signal performance, e.g., to improve signal strength).

Collection and delivery module 110 can be placed near collection zone 116 (e.g., in the vicinity of collection zone 116). Collection and delivery module 110 may be in contact with collection zone 116 (e.g., a specific region of skin) or it may be separated from the collection surface 118 by a spacer.

Collection zone 116 and adjacent collection structures 112 may be surrounded by a sealant material or a sealing structure 210 to ensure that most or all of the fluid cannot leak out or evaporate. For example, sealant 210 may ensure that the only possible direction of fluid transport is through collection structure 112 of collection and delivery module 110 (e.g., an O-ring may surround the perimeter of collection zone 116). Sealant 210 may include a sealant material that is semi-permeable (e.g., impermeable to a liquid of interest and permeable to air). Sealant 210 may act as a spacer layer defining a distance between collection zone 116 and collection structures 112. It may also act as a fixation system. It may include of an elastomer, gel, grease, glue (e.g., silicone or acrylate glue), an adhesive (e.g., a skin adhesive), a laminate (e.g., an adhesive trilaminate).

For fluid that emerges to a surface via pores or ducts (e.g., for a fluid such as sweat), collection structures 112 are dimensioned (e.g., sized and shaped) such that they address a plurality of pores or ducts (e.g., at least one pore or duct is addressed by the collection, e.g., a plurality of pores and ducts are addressed). For example, the density of sweat ducts on human skin is in a range from about 0.1 to about 10 ducts per mm² depending on the location on the body. In certain embodiments, collection zone 116 may have a surface area of greater than 10 mm² (e.g., to address at least one pore, e.g., at the lowest pore density of about 0.1 pore per mm²). In other embodiments, the surface is larger to address greater than one pore.

For fluid in the form of drops on a surface, collection structures 112 are designed to collect fluid from the drops. Collection structure 112 typically includes at least one fluidic channel or a fluidic channel network 114.

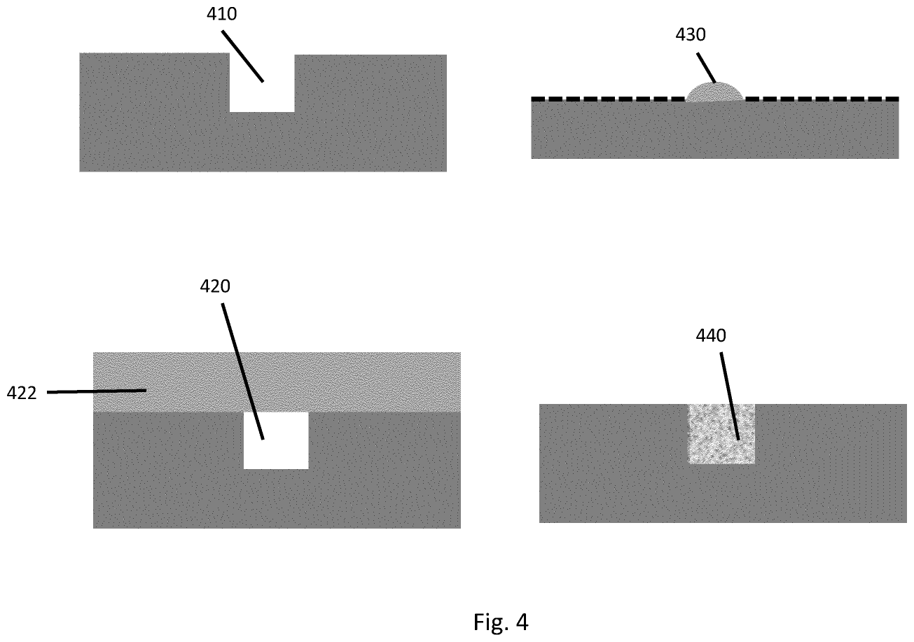

FIG. 4 shows cross-sections of fluidic channels. As used herein, a “fluidic channel” may refer to the following implementations (or a combination thereof): an open channel such as a groove 410 in the collection zone surface, an closed microfluidic channel 420 (e.g., closed with a laminated lid 422), a “two-dimensional” (“2D”) channel 430 defined by surface properties [e.g., where the channel is on a surface (e.g., does not include side walls and a top) and is defined by surface energy contrast (such as hydrophilic/hydrophilic patterning)], or a channel including a fixed gel matrix 440 that is permeable to the fluid (e.g., a groove or microfluidic channel filled with a cross-linked hydrogel that is permeable to water).

A channel comprising a gel matrix typically has a high affinity for fluid and can prevent unwanted drying of the channel (e.g., a channel filled with a hydrogel may dry slower than a channel filled with water because of the hydrophilicity of the hydrogel).

A fluidic channel (e.g., or a fluidic channel network) may be fabricated by one or a combination of the following techniques (and possibly as multilayers of one or a combination of techniques): photolithography of a photosensitive polymer such as SU-8

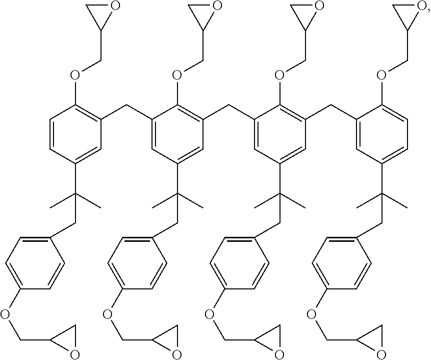 [SMILES: CC(C)(Cc1ccc(OCC2CO2)cc1)c1ccc(OCC2CO2)c(Cc2cc(C(C)(C)Cc3ccc(OCC4CO4)cc3)cc(Cc3cc(C(C)(C)Cc4ccc(OCC5CO5)cc4)cc(Cc4cc(C(C)(C)Cc5ccc(OCC6CO6)cc5)ccc4OCC4CO4)c3OCC3CO3)c2OCC2CO2)c1]

by application of a laminated plastic foil patterned by photolithography or pre-patterned (e.g., by imprinting) or pre-cut (e.g., by laser), using plastic injection, and with the grafting or deposition of a gel.

A fluidic channel (e.g., or a fluidic channel network) may include a combination of open channels (e.g., inlets where fluid is collected) and of closed channel (e.g., through which the fluid is delivered to a sensor). In some embodiments, this is achieved using a double layer of independently structured SU-8 layers bonded together. In other embodiments, this is achieved by locally covering open channels (e.g., made of a single layer of patterned SU-8) with a laminated polymer film that is affixed to the open channel layer with an adhesive (e.g., tape). The adhesive can be hydrophilic.

The surface properties (e.g., the surface energy) of the fluidic channel or channel network may be tuned by physical and/or chemical treatment(s) (e.g., to render the channel hydrophilic) to promote the wetting and filling of the channel by capillary action. This may be performed by surface activation (e.g., with oxygen plasma), by functionalization (e.g., with specific molecules), by grafting of a functional molecule to the surface (e.g., a self-assembled monolayer, e.g., of one or more silanes and/or one or more thiol(s)). A gel (e.g., hydrogel) coating can also be grafted to or coated on the surface of the fluidic channel(s).

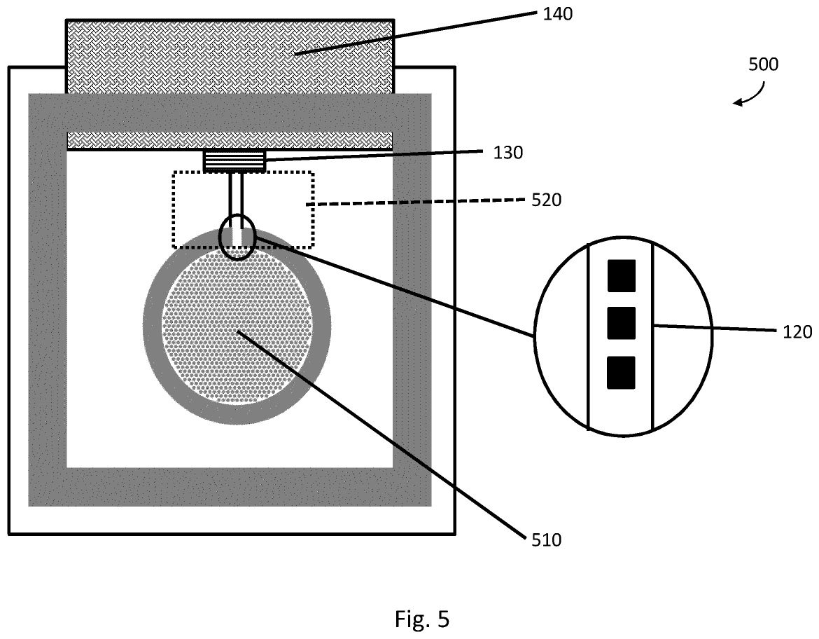

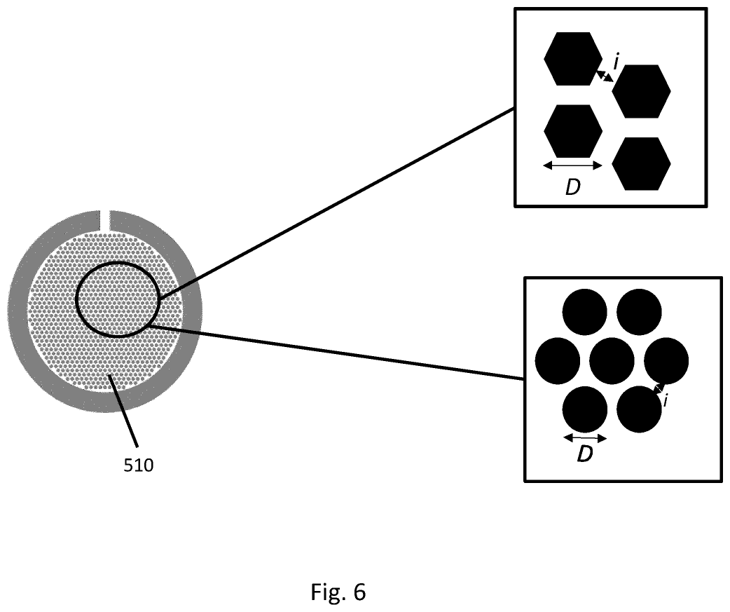

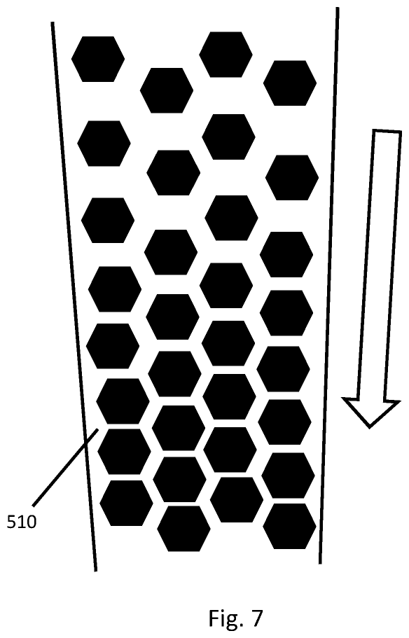

A fluidic channel may include pillars or pavement structures (or arrays thereof) to reduce dead volume in the channels and to facilitate fluid transport via capillary action. FIGS. 5 and 6 show illustrative examples of a system 500 that include a microfluidic collection and delivery module 110 with pillars-based collection structures 510. In certain embodiments, collection and delivery module 110 includes an optional lid 520 locally closing the channels. The pillars (e.g., or pavement) can have two primary parameters: the pillar diameter D and the inter-pillar distance i. For sweat collection on skin, for example, the pillar diameter D and inter-pillar distance i are in a range between 1 μm to 1 mm. In certain embodiments, the pillar diameter D is in a range from about 10 μm to about 800 μm, or from about 100 μm to about 500 μm. In certain embodiments, the inter-pillar distance i is in a range from about 10 μm to about 800 μm, or from about 25 μm to about 100 μm. Each pillar array may be designed such that they have a size or interstitial distance gradient in one direction to promote directional flow towards the sensor, as shown in the illustrative example of FIG. 7.

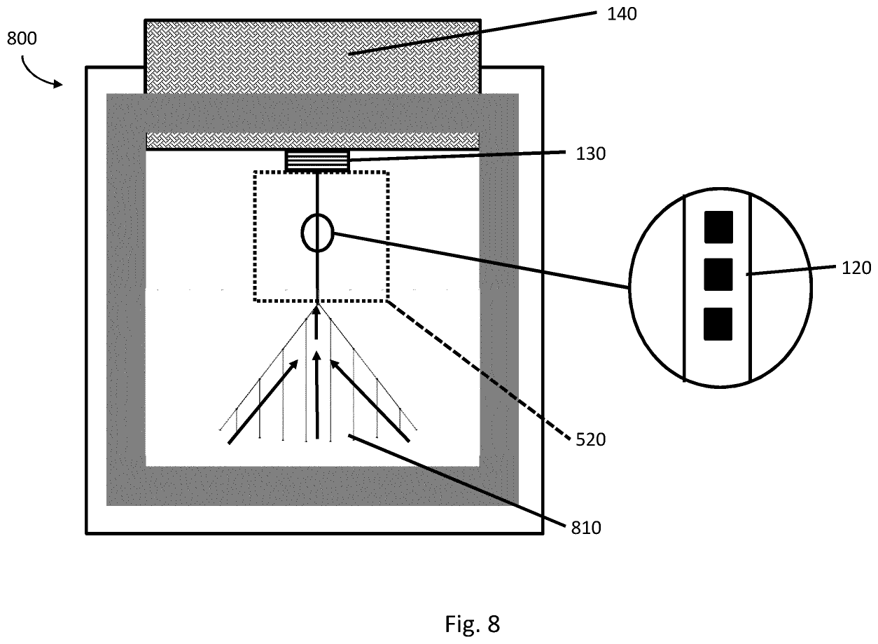

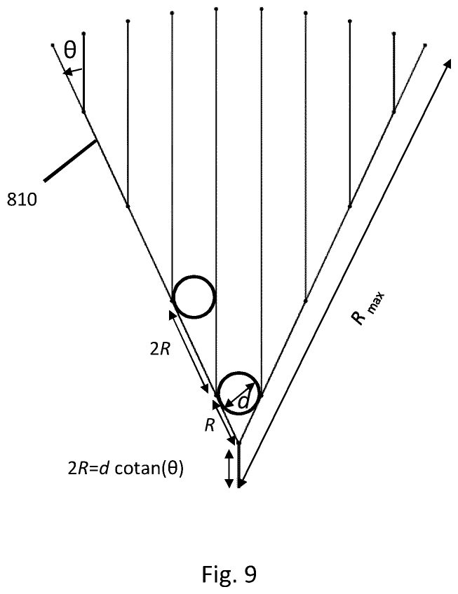

As in the system 800 shown in FIG. 8, in certain embodiments, the fluidic channel network includes arborescent collection structures and delivery channels 810 with branches that address the surface (e.g., with a regular spacing, e.g., corresponding to the spacing of pores on the skin) of collection zone 116 (e.g., the branches may have biomimetic and/or a fractal geometry). Each channel (e.g., or “branch”) of arborescent collection structures and delivery channels 810 may capture (e.g., collect) fluid to be delivered to main sensor module 120, as shown in the illustrative examples of FIGS. 8 and 9. Arborescent collection structures and delivery channels 810 (e.g., or other arborescent structures) can be characterized by a “tree opening” angle ϑ, a maximum extent of the “tree” (e.g., a tree radius) Rₘₐₓ, and an inter-branch distance d.

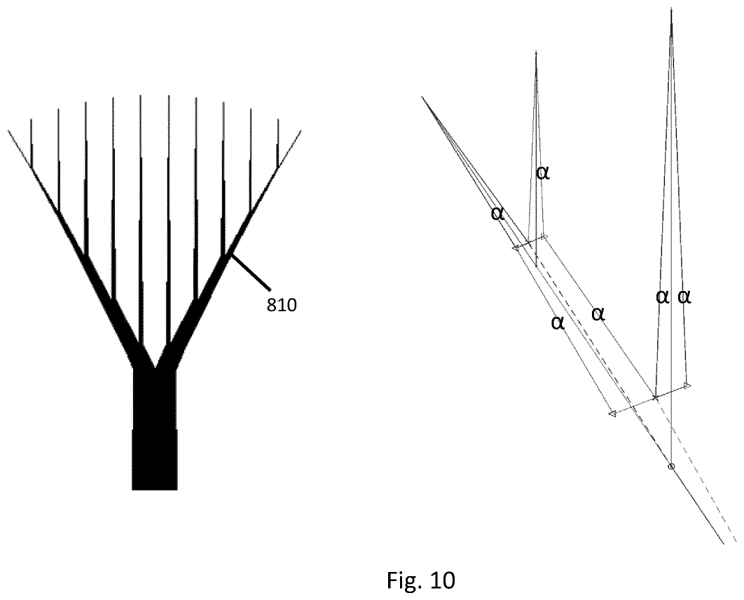

The branches of the channels 810 may be designed to provide a constant opening angle to promote filling by capillary action. An example of this is shown in the illustrative example of FIG. 10. FIG. 10 shows the same structure presented in FIG. 9 but with an additional parameter: a constant opening angle (a). For example, a “2D” channel may be defined by differences in surface properties (e.g., surface energy) (e.g., using hydrophilic/hydrophilic patterning).

In certain embodiments, fluid emerges from a surface (e.g., a wet surface, e.g., skin) as droplets. For example, fluid can emerge as droplets from ducts in the surface of skin. For collecting drops arborescent collection structures 810 (with branches separated by the inter-branch distance d) can be used. Arborescent collection structures 810 can be separated from the collection zone 116 by a spacer layer (e.g., a sealant, e.g., an adhesive) with a thickness e. In general, it is not possible to align the pores (e.g., of the skin) with the branches of arborescent collection structures 810. For example, alignment of the collection structures 810 to the pores in skin may not be practical. For example, the substrate on which the collection structures 810 are fabricated may not be transparent, preventing practical alignment of the collection structures 810 to the pores. In certain embodiments, in order to be collected, a droplet must grow until it reaches a threshold height that is determined from the spacer layer thickness e. The droplet may then need to grow laterally until the surface of the droplet reaches at least one of collection structure 810.

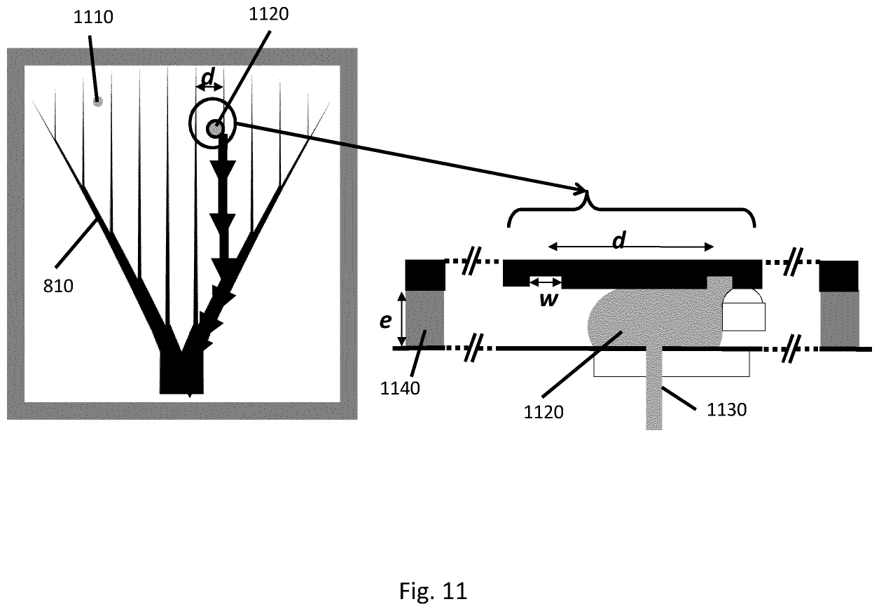

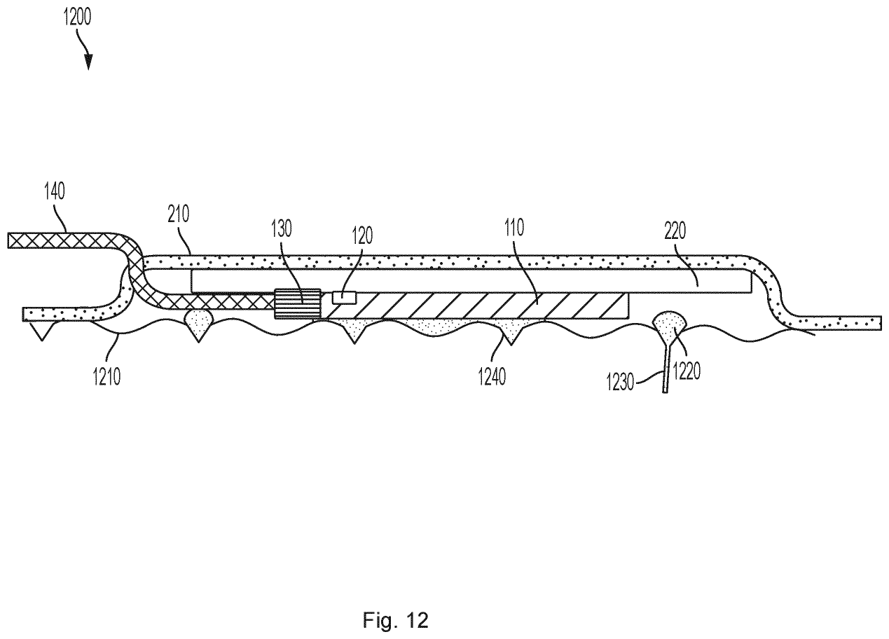

In certain embodiments, the maximum lateral size a droplet achieves once touching the surface of the collection zone is half the inter-branch distance d minus half the channel width w (i.e., d/2-w/2). The global dead-volume of the fluidic channel network can be increased by decreasing the inter-branch distance d and/or increasing the channel width w. In certain embodiments, the time required to transport a fluid from collection surface 118 (e.g., the surface of collection and delivery module 110) to main sensor module 120 requires a tradeoff to determine an optimum inter-branches distance d and the channel width w. The values may be selected based on a given application. For example, the spacer layer e may be designed to be as thin as possible as long as it remains functional (e.g., as a sealant, e.g., as an adhesive). FIG. 11 shows an illustrative example of a branch of arborescent collection structures and delivery channels 810. If the lateral dimension of a droplet 1110 is too small, it cannot reach one of collection structure 810 and will not be collected. Once the size of a droplet 1120 coming from a pore or duct 1130 reaches the lateral size (i.e., d/2-w/2), it can be collected. For fluid collection on skin, the distance d is may be in a range from about 50 μm to about 1 mm. The width of the channel may be in a range from about 1 μm to about 1 mm. When a spacer layer 1140 is present, the thickness (e) may be, for example, in a range from about 10 μm to about 500 μm. For fluid collection via collection structures on a surface that has a v-groove network (e.g., certain regions of human skin), the v-grove network itself may participate to fluid transport. This may particularly be the case in embodiments where the collection structure is applied directly to the surface (e.g., without a spacer layer, e.g., with a thin spacer layer). In this embodiment, a sealant (e.g., an adhesive, e.g. a laminate) may be applied over the edge of the collection structure (e.g., to cover both the edge of the collection structure and the surface on which the fluid is collected). FIG. 12 shows an illustrative example of a system for collection and analysis of a fluid. The system is affixed to and in direct contact with the skin 1210 by a sealant 210. A portion of sweat droplets 1220 coming from sweat ducts 1230 can spread within the v-groove network 1240 of the skin 1210 and be transported by the v-groove network 1240.

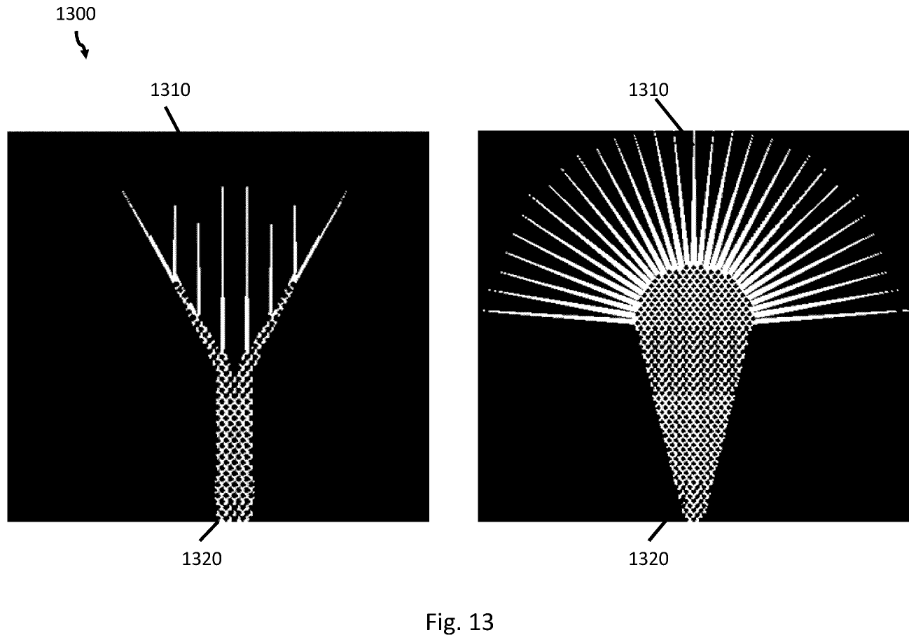

FIG. 13 is a schematic diagram showing hybrid collection structures 1300. In certain embodiments, a channel network includes both arborescent collection structures 1310 and pillar-based collection structure 1320. In certain embodiments, the arborescent structures address the surface where sweat droplets can randomly appear more efficiently (e.g., with low dead volume). In certain embodiments, pillars structures have more dead volume, however they are more robust to clogging (e.g., as fluid can find alternative path to flow if one interstice get clogged). Therefore it may be beneficial to have hybrid collection structures in which the arborescent structures (e.g., “branches”) merge to the pillar-based structures (e.g., “trunk”) so that in the event of clogging the trunk can still be able to carry liquid over even if some branches may be lost due to clogging.

A filter may be added to channel network 114 to exclude contaminants, depending on the application. For sweat collection, contaminants may be lipids, bacteria, particles, and/or dead skin cells. The filter may include an ensemble of obstacles that would sterically exclude (e.g., exclude based on size) the contaminants (such as an array of pillars structures, or a fiber matrix such as paper or a textile). The filter may also include a gel (e.g., a hydrogel) matrix. In certain embodiments, the pillars structures may act as a filter (e.g., if some or all of the pillar-filled fluidic channel includes a locally closed channel, e.g., the channel is closed with a lid, e.g. with a lid comprising a laminated film), for example, as depicted in the illustrative example of FIG. 13.

Collection and delivery module 110 is typically designed to optimize the delay (e.g., make the delay short, e.g., make the delay coincide with a desired frequency of measurements) between the emergence of the fluid at the collection zone and the readout by main sensor module 120. This, for example, improves the time response of the sensor. For readouts that are sensitive to diffusion (e.g., based on diffusion-limited process) (e.g., temperature and concentration measurements), this approach decreased the unwanted effects of averaging out (e.g., dilution/diffusion) of information during diffusive transport.

Collection and delivery module 110 may be designed to prevent bubbles (e.g., air bubbles) from being trapped in the system (e.g., in the fluidic channels). In order to avoid bubbles from being trapped, the fluidic channel network 114 may direct (e.g., funnel) fluid to a single channel before the fluid is delivered to main sensor module 120. Serpentine channels may intersect all the sensors serially because bubbles were found to be trapped within a branch of a Y-junction, preventing bubbles from obstructing measurements.

A serpentine structure may also be used to maximize the overlap of a fluidic channel (e.g., a microchannel) with the surface of a sensor, particularly, for example, when the sensor is wider than the fluidic channel (e.g., a disk-shaped reference electrode with a diameter greater than the width the fluidic channel).

### Main Sensor Module

Chemical and/or physical analysis is performed by main sensor module 120. This analysis may, for example, include the measurement of the concentration of substances present in the fluid, the pH, the conductance, temperature, the pressure, flow rate, the velocity of the fluid. The substances measured by the sensor may be ions (e.g., chloride, sodium, potassium), sugars (e.g., glucose), biomolecules (e.g., polynucleotides, proteins, hormones, enzymes, antigens, neuropeptides, antibodies), and any other solute.

A sensor in main sensor module 120 may be based on a physical and/or chemical principle. For example, a sensor may use an electrochemical, electrical, or optical signal. a sensor may be based on an electrode, based on a semiconducting element (e.g., a capacitor e.g., a MOSCAP) or a transistor.

The measurement of pH or of the concentration of a substance may be based on a field-effect transistor (e.g., an ion-sensitive field-effect transistor, (ISFET)) or the electrochemical response of an electrode (e.g., the measurement of an open-circuit potential, a voltammetric measurement, an amperometric measurement, or an impedance measurement). The surface a sensor may be functionalized to detect a specific substance.

In certain embodiments, the sensors in main sensor module 120 may include one or more arrays of field effect transistors (FETs) (e.g., ion sensitive FETS (ISFETs), e.g., fully depleted FETs (FD-FETs)). The array(s) of FETs may include FETs with a ribbon architecture fabricated on a fully depleted silicon-on-insulator substrate with a buried oxide layer (an FD-SOI substrate). The present disclosure encompasses the recognition that the dimensions and design of the FD-SOI substrate allows for devices with less complex fabrication processes, improved electrostatic control of the FET, a decreased parasitic capacitance between source and drain, decreased leakage currents, and decreased power consumption compared to previous technology.

The FD-SOI substrate allows FD-FETs to be fabricated with a ribbon-like structure with less strict dimensional constraints. Thus, FD-FET sensors can be fabricated with a larger sensing surface area (e.g., for improved sensor signal) than was possible using previous approaches, while maintaining the excellent electrical properties of the FD-FET. For example, the surface area of the gate of the semiconductor sensor (e.g., an FD-FET sensor) can be in a range from about 1 μm² to about 1000 μm² or larger. In certain embodiments, the surface area of the gate of the semiconductor sensor (e.g., an FD-FET sensor) is in a range from about 35 μm² to about 150 μm².

In certain embodiments, the FET sensors have liquid gates that are functionalized for the detection of selected biomarkers. For example, one or more of the sensors may have a gate that includes hafnium dioxide (HfO₂) (e.g., for use as a pH sensor). Each sensor (e.g., the gate of each FET) is functionalized to detect and/or quantify a biomarker of interest.

Examples of sensors which may be used in the systems, methods, devices, apparatus, and architectures presented herein are described in European Patent Application No. 16188227.9 filed Sep. 10, 2016, U.S. patent application Ser. No. 15/453,920 filed Mar. 9, 2017, International Patent Application No. PCT/IB2017/055456 filed Sep. 11, 2017, and U.S. patent application Ser. No. 15/913,714 filed Mar. 6, 2018, International Patent Application No. PCT/EP2018/077793 filed Oct. 11, 2018, the contents of each of which are incorporated herein in their entirety.

Main sensor module 120 can also include sensors for measuring other properties or the environment. For example, main sensor module 120 may include a temperature sensor, a flow rate sensor, a conductance sensor, an ionic strength sensor, a pressure sensor, and/or a pH sensor.

A conductance or ionic strength sensor may be based on the impedance readout of a pair of electrodes (e.g., platinum electrodes or Ag/AgCl electrodes).

The temperature sensor may be based on a thermocouple, thermistor, a diode-based temperature sensor or a resistance temperature detector (RTD).

The pressure sensor may be a gauge-based pressure sensor, a MEMS based pressure sensor, a piezoresistive pressure sensor.

A flow rate sensor may sense the flow rate and/or the velocity of the fluid based on heat transfer detection (e.g., a calorimetric flow meter, hot wire flow meter, time-of-flight flow meter). A capacitive flow sensor may be used to measure flow rate based on the change of impedance of an electrode. An electrokinetic flow rate sensor may be used to measure flow measurement using measurements of streaming potential. An acoustic or optical flow meter may be used to measure flow rate based on the Doppler effect. A flow meter may also measure flow rate based on differential pressure measurements.

Main sensor module 120 may also include a reference device, such as a non-functionalized instance of a sensor to allow for differential measurements.

Main sensor module 120 may also include a reference electrode producing a stable reference voltage (such as a silver-silver chloride electrode).

The sensors of main sensor module 120 may be functionalized to sense a specific molecule. The functionalization may be based on at least one of the following techniques: the deposition of a thin film on the surface of the sensor, a functional molecule grafted to the surface of the sensor, and/or a functional membrane acting as a selective barrier for the molecule to be sensed. Functional thin films of interest may be dielectrics and/or metal, for instance hafnia, silver chloride, and iridium oxide. A functional molecule grafted on the sensor may be for instance a self-assembled monolayer. A functional membrane may be for instance an ion selective membrane or a functionalized polymer matrix.

A polymer layer or gel (e.g., hydrogel) layer may be deposited or grafted on top of a sensor for at least one of the following purposes: to prevent unwanted drying of the sensor (e.g., a hydrogel has a high affinity for water), to physically protect the sensor (e.g., protection from damage by contact or from electrostatic discharge), to filter out a contaminant (e.g., for sweat collection, lipids, bacteria, particles, and/or dead skin cells may be filtered out), to facilitate sensing via a high affinity for a substance of interest that is being sensed (e.g., a substance of interest may have a higher partition coefficient in a gel relative to in the fluid) and/or to act as a selective barrier that may exclude some substances (e.g., such substance has a lower partition coefficient in the gel relative to the fluid) that may interfere with the functioning of the sensor (e.g., impacting the cross-sensitivity to the substance being sensed). For example, negatively charged species may be enriched and positively charged species may be depleted within a positively charged hydrogel. These properties may be used to improve sensing of a specific charged substance.

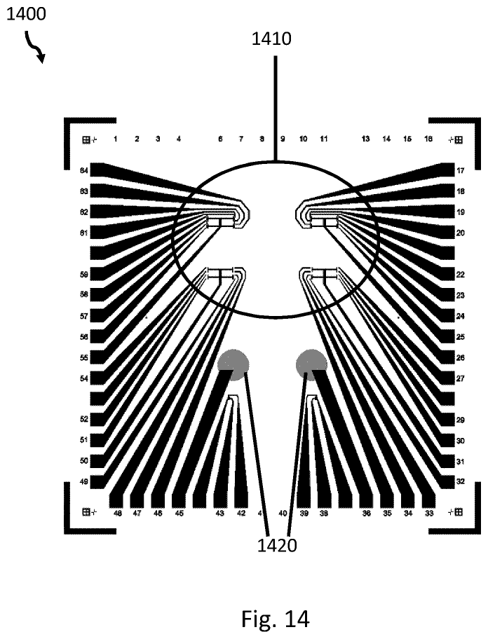

FIG. 14 shows an illustrative example of main sensor module 1400 that includes 32 ISFET sensors 1410 and 2 reference electrodes 1420.

### Flow Regulation Module

Flow regulation module 130, shown in FIGS. 1-3, controls the fluid flow in order to optimize the operation and response time of main sensor module 120. Flow regulation module 130 is designed to optimize the delay time between the emergence of the fluid on the collection zone and the readout by the sensors, while preventing the drying out of the collection and delivery system and/or the sensors surface. Flow regulation module 130 may be passive or active (e.g., actuated by a powered pump, e.g., controlled by an electronic device connected to the system).

Flow regulation module 130 comprises at least one of the following components: a capillary pump [e.g., comprising an array of pillars or pavements and/or a wicking or absorbent material (e.g., a paper or a textile)], a patterned surface [e.g., with a particular surface property (e.g., surface energy)], a barrier (e.g., a hydrophobic zone for flow of a water-based solution), and/or a fluidic valve.

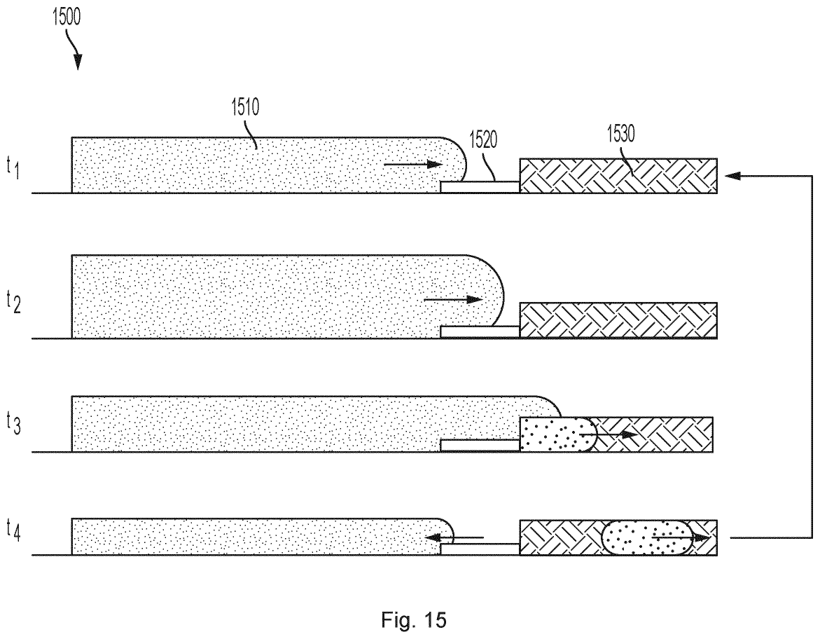

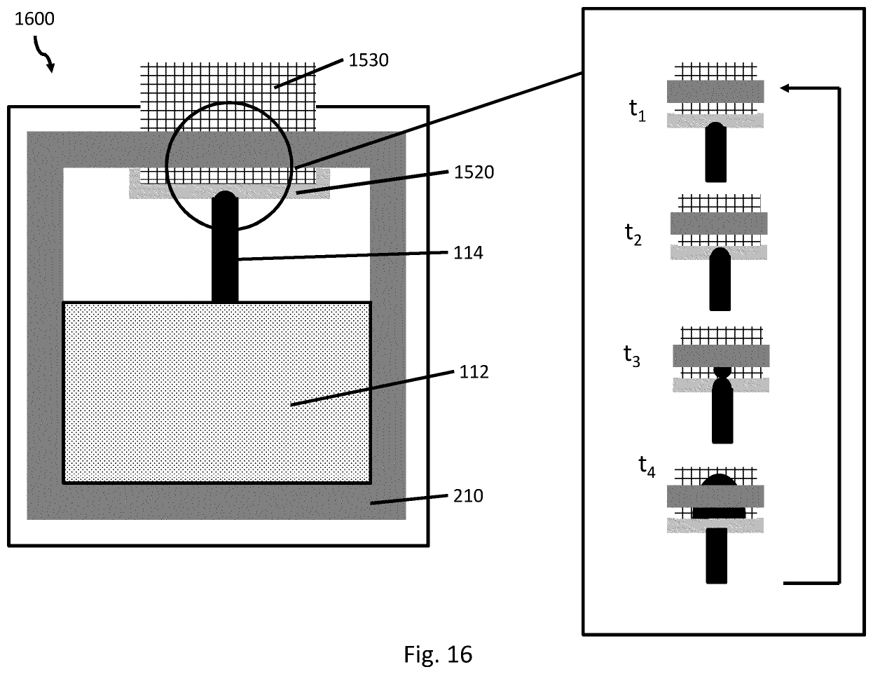

Flow regulation module 130 may be a passive flow regulation module. A passive flow regulation module may be an overflow control device based on the combination of a surface patterned with a give surface property (e.g., surface energy) (e.g., that provides a flow barrier) and a high flow rate (e.g., high absorption rate) capillary pump. An overflow control device is dimensioned (e.g., shaped and sized) such that the surface property (e.g., surface energy) barrier always guarantees that collection and delivery module 110 and main sensor module 120 remain wet even at a low or zero flow rate of fluid from the collection zone (after initial wetting). Meanwhile, the capillary pump is dimensioned (e.g., shaped and sized) to absorb the fluid emerging in the collection zone even at maximum flow rate. FIGS. 15 and 16 show illustrative examples of such an overflow device.

FIG. 15 is a schematic diagram showing a flow regulation process 1500 by flow regulation module with a passive overflow device. In certain embodiment, flow regulation module includes a surface property barrier 1520 and a wicking material or capillary pump 1530. At t₁, a fluid 1510 flowing from main sensor module or collection and delivery module reaches a surface property barrier 1520 (e.g., a hydrophobic barrier). At t₂, as the volume of the fluid 1510 grows, the fluid passes surface property barrier 1520 and approaches a wicking material or capillary pump 1530. At t₃, when the fluid 1510 reaches the wicking material or capillary pump 1530, part of the fluid is absorbed by the wicking material 1530, or transported by the capillary pump. At t₄, the fluid 1510 breaks into two parts, with one part transported by the wicking material or capillary pump 1530 and the other part flowing back.

FIG. 16 is a schematic diagram showing a top view of a system 1600 for collection and analysis of a fluid from a surface with a flow regulation module, according to illustrative embodiments.

An active flow regulation module may be based on an actuated fluidic valve such as a mechanical valve, a pneumatic valve, a hydraulic valve or an electrovalve (e.g., based on the control of the electrowetting of an interface or based on the control of electroosmotic flow).

In certain embodiments, flow regulation module 130 is positioned (e.g., implemented) between main sensor module 120 and waste module 140. In other embodiments, flow regulation module 130 is positioned between collection and delivery module 110 and main sensor module 120, or embedded within main sensor module 120.

### Waste Module

Waste module 140 is designed to collect and/or dispose of the fluid after analysis. It may be based on a capillary pump, for instance an array of pillars (e.g. hexagonal) or pavements and/or of a wicking material based for instance on paper, textile, gel or an absorbent material. This pump or wicking material may be in turn connected to an absorbent pad acting as a waste reservoir. The reservoir may be dimensioned (e.g., sized and shaped) so that it is never full (e.g., the rate of evaporation is equal to or greater than the rate of waste collection). Thus, in certain embodiments, fluid collection can proceed continuously (e.g., over long period of time without interruption). Waste module 140 may be designed in such way as to have one end promoting fluid evaporation.

Waste module 140 may include a wicking material that is mounted at the outlet of any of the modules described herein (e.g., such that all exiting fluid is collected by the wicking material). In certain embodiments, the wicking material may be installed on the same plane as any of the modules described herein. For example, the wicking material may be separated from the surface of the collection zone by the sealant material.

### Wetting Sensor Module

Wetting sensor module 150 may indicate if some or all of the above-mentioned modules are wet and provide an estimate of the flow rate during the filling of the system. It may, for example, include a series of electrodes installed in some or all of the modules including 110, 120, 130 and 140 (and possibly more than one per module). By performing conductance measurements between pairs of electrodes, it is possible to measure whether there is ionic contact between them and hence whether the path between them is wet. By using various combinations of electrodes, it is thus possible to track the fluid progression along the way, and hence to compute an estimate of the flow rate using the known geometry and the fluidic capacity of the system.

Some of the electrodes of wetting sensor module 150 may be installed face-to-face as pairs. The electrodes may comprise a noble metal and be actuated in alternating current (AC) (e.g., in the 1 to 100 kHz range). The electrodes may be also actuated in direct current (DC) [e.g., where a voltage is applied with reference to (e.g., versus) a silver/silver chloride (Ag/AgCl) electrode(s)]. Electrodes of wetting sensor module 150 may include one or more metal, (e.g. a noble metal, e.g. platinum or Ag/AgCl), conductive ink, or conductive polymer electrodes.

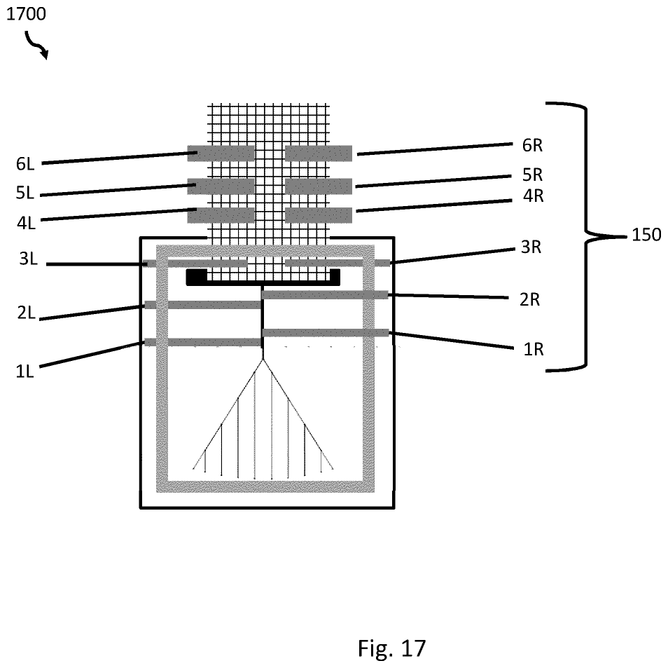

FIG. 17 shows an illustrative example of a system 1700 equipped with wetting sensor module 150. In certain embodiments, wetting sensor module 150 includes six pairs of electrodes (1L-1R, 2L-2R, 3L-3R, 4L-4R, 5L-5R and 6L-6R) located in modules 110, 120, 130 and 140. As shown in FIG. 17, electrodes 1L and 1R are disposed in collection and delivery module 110. Electrodes 2L and 2R are disposed in main sensor module 120. Electrodes 3L and 3R are disposed in flow regulation module 130. Electrodes 4L, 4R, 5L, 5R, 6L and 6R are located in waste module 140.

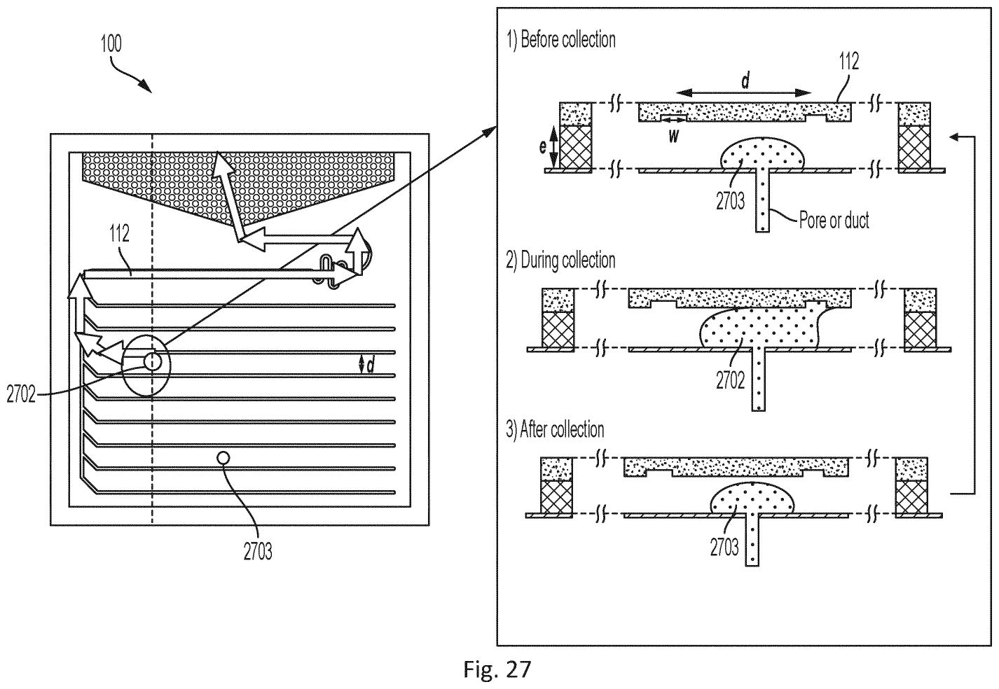

FIG. 27 is a schematic diagram showing a top view and a cross-section view of a system for collection of a fluid (e.g., a biofluid, e.g., sweat) from a surface (e.g., skin of a user) by a hydrophilic channel network, according to some embodiments. A surface (e.g., skin) can become wet (e.g., or semi-wet) by fluid from, for example, condensation, diffusion, permeation through pores and/or ducts in a person's skin. The fluid may be a biofluid, such as sweat emerging from sweat ducts on the surface of a person's skin. In certain embodiments, collection and delivery module 110 of wearable system 100 includes a substrate patterned with structures (e.g., collection structures) that are designed to collect fluid (e.g., a biofluid, e.g. sweat) on another wet (e.g., semi-wet) surface (e.g., skin) that is in contact with (e.g., substantially in contact with) the substrate with the collection structures 112. Fluid is collected in a collection zone of the collection surface (e.g., a region of skin addressed by the substrate of the device) using collection structures 112 and directed through one or more fluidic channels. In certain embodiments, fluid flow is driven passively (e.g., by capillary action). In other embodiments, active flow is used (e.g., driven by one or more fluid pumps, e.g., pumps requiring power).

In certain embodiments, fluid emerges from a surface (e.g., a wet surface, e.g., skin) as droplets. For example, fluid can emerge as droplets from ducts in the surface of skin. Arborescent collection structures (with branches separated by the inter-branch distance d) can be used for collecting drops. In certain embodiments, as illustrated in FIG. 27, in order to be collected, a droplet 2703 must grow until it reaches a threshold height that is determined from the spacer layer thickness e. The droplet 2703 may then need to grow laterally until the surface of the droplet 2703 reaches at least one of the collection structures 112 (e.g., as with droplet 2702), shown in the cross-section view of FIG. 27.

If the lateral dimension of a droplet is too small, it cannot reach one of the collection structures 112 and will not be collected. In certain embodiments, the arborescent structures address the surface where sweat droplets can randomly appear more efficiently (e.g., with low dead volume).

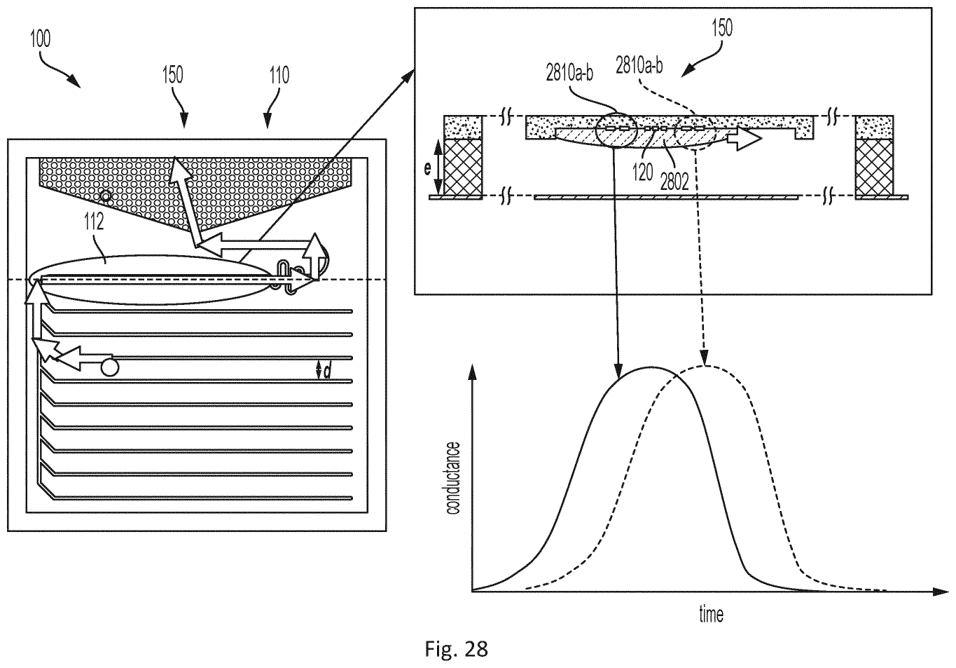

FIG. 28 is a schematic diagram showing a top view and a cross-section view of a wearable system 100 comprising a fluid collection and delivery module 110 and a wetting sensor module 150. A collected droplet 2802 is travelling along a sensor delivery channel 112. Wetting sensor module 150 may indicate if one or more modules included in wearable system 100, such as fluid collection and delivery module 110, are wet and provide an estimate of the flow rate during the filling of the system. It may, for example, include one or more sweat presence monitoring devices including a series of electrodes installed in some or all of the modules including 110, 120, and 130 (and possibly more than one per module). In the example of FIG. 28, two sweat presence monitoring devices 2810a-b are included, each including a respective pair of sensing elements that, in this case, are electrically conductive (e.g., metal) electrodes. In some embodiments, two sweat presence monitoring devices 2810a-b share a common sensing element (e.g., and each include at least one non-common sensing element). In some embodiments, sensing elements of two sweat presence monitoring devices 2810a-b are interdigitated (not shown in FIG. 28). By performing conductance measurements between pairs of sensing elements, it is possible to measure whether there is ionic contact due to a conductive pathway between them and hence whether the path between them is wet. In some embodiments, optical sensing elements are used, for example that measure fluorescence of a biofluid, if present. By using various combinations of electrodes, it is thus possible to track the fluid progression along the way, and hence to compute an estimate of the flow rate using the known geometry and the fluidic capacity of the system.

Some of the electrodes of a wetting sensor module 150 may be installed face-to-face as pairs. The electrodes may comprise a noble metal and be actuated in alternating current (AC) (e.g., in the 1 to 100 kHz range). The electrodes may be also actuated in direct current (DC) [e.g., where a voltage is applied with reference to (e.g., versus) a silver/silver chloride (Ag/AgCl) electrode(s)].

Referring still to FIG. 28, each sweat presence monitoring device 2810a-b can be used to monitor a drop 2802 travelling through a wearable 100 (past a main sensor module 120 as shown by the black arrow on the leading edge of the drop 2802). Depending on factors, including placement and local geometry, an output of conductance signals of each sweat presence monitoring device 2810a-b may follow a substantially identical curve, though with some characteristic temporal phase shift between the curves such that a sweat rate can be determined based on a change in the signals output by the sweat presence monitoring devices 2810a-b (in this example, based specifically on the temporal phase shift). Where sweat rate is low to moderate (S1), each drop can be individually detected and monitored. If the sweat rate is too high (S2), sweat will be continuously present, which will mean that signal from the sweat presence monitoring devices 2810a-b will be continuous and unable to accurately discern a sweat rate. Further embodiments are described subsequently that can facilitate sweat rate determination even at high sweat rates.

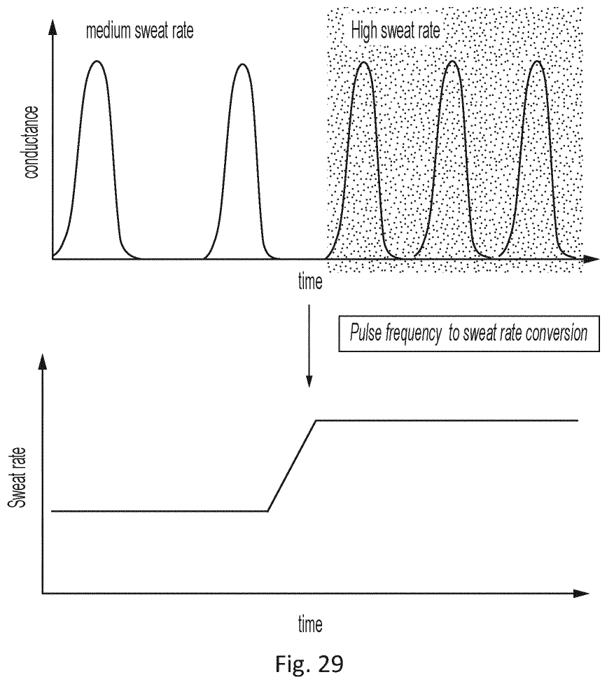

FIG. 29 is a schematic diagram showing the conductance readout as a function of time and corresponding sweat rate in a single pore scenario, according to illustrative embodiments. When a droplet is collected, it then travels through a wearable system 100, for example from a fluid collection and delivery module 110, to a main sensing module 120, until it reaches the outlet, still as a droplet, until the next droplet emerges from the sweat duct and is collected, and so on. Therefore, the fluid flows through the whole microfluidic system drop after drop in a pulsatile way, each sweat duct releasing a droplet periodically, which is then collected and processed by wearable system 100. In this situation, a sweat presence monitoring device (e.g., comprising electrodes forming a conductance sensor) will also give a pulsatile signal corresponding to the passing of the droplets on the sensor, each pulse corresponding to one droplet. Once a drop of fluid gets in contact with, for example a fluidic collection zone of a fluid collection and delivery module 110, it spreads and fills in the whole channel 112, resulting in a certain level of fluid inside the channel 112. The signal intensity rises within this phase. When the fluid front gets in touch with, for example, a drain material of waste module 140, due to the fact that the drain material has a higher hydrophilicity than the channel 112, most of the fluid spread in the channel in phase 1 will be absorbed by the drain material. The signal intensity drops within this phase. Since the droplet size can be estimated, it is possible to get an estimate of the sweat rate on the collection zone from the analysis of the conductance change over time as measured by electrodes of the sweat presence monitoring device.

The relation linking conductance change over time to sweat rate is simpler at lower (e.g., low to moderate) sweat rate (S1), when the situation where two sweat droplets are collected in the same time is extremely unlikely (the arborescent collection structure address more a plurality of pores). As the sweat rate increases, this situation gets more likely to occur and a more complex model is developed to link conductance and sweat rate. With the periodic alternating of the two above described phases, it is possible to analyze the fluid generation rate with signal processing methods.

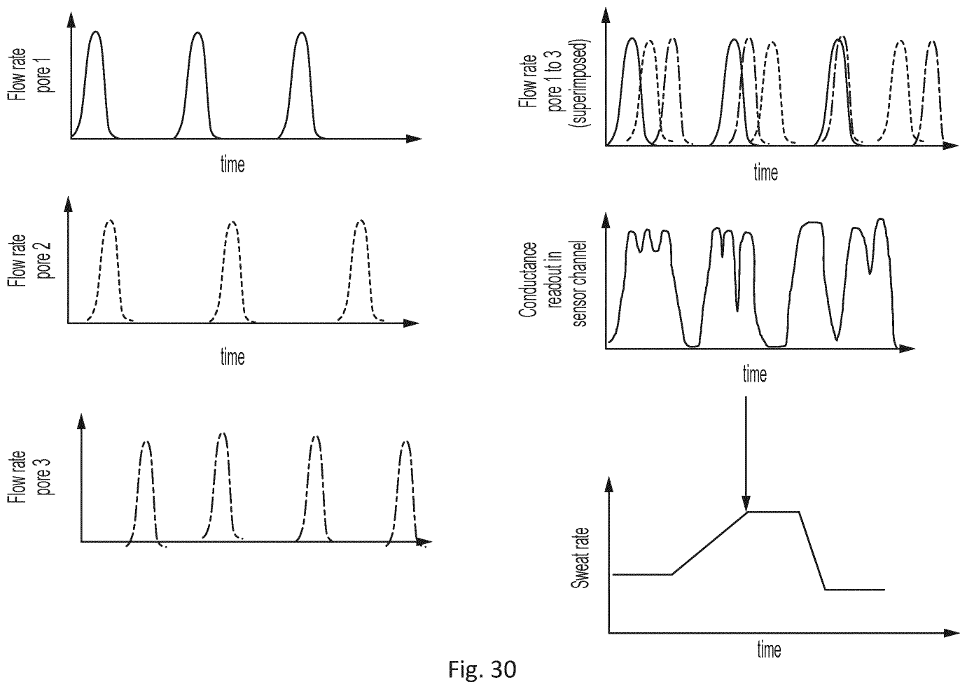

FIG. 30 is a schematic diagram showing the conductance readout as a function of time and corresponding sweat rate in a multiple pores scenario, according to illustrative embodiments. The frequency of this periodic alternating behavior will be proportional to the fluid generation rate. As shown, multiple pores can have slightly different sweat generation rates, each being a periodic generation of drops of sweat. Superimposing the individual sweat generation curves of the multiple pores yields a complex conductance readout from a sweat presence monitoring device. Therefore, frequency spectrum analysis method (e.g. fast Fourier transform, spectrogram or FFT based time-frequency analysis), or period measuring techniques (e.g. rising/fall edge detection) can be used to extract a fluid generation (sweat) rate, as shown in the bottom right.

In case that a single drop of fluid is generated continuously, if the fluidic properties are constant, the volume of drop that is spread in the channel in each period will also be constant, and so is the time that the signal intensity stays high as well as the time that the signal intensity stays low. This will result in a pulse-width-modulated signal with constant duty cycle, constant time for the high or low sections of each period.

If there are multiple sources of fluid generator (e.g., pores) in contact with the same fluidic structure 112, but not synchronized when generating fluid flow. Now that if the interest is the total or average fluid generation rate, we can still leverage the properties, e.g., when the fluidic properties are constant, the duty cycle will still be constant.

Since the intensity of the signal will be proportional to the level of fluid inside the fluidic channel, an integration of the signal over time (tₜ) will be proportional to the total volume (Vₜ) of fluid that travelled through the channel during tₜ. Therefore the fluid generation (sweat) rate can be simply calculated as Vₜ/tₜ.

In some embodiments, sweat is generated by a subject (e.g., human) and collected by wearable system 100, for example in fluid collection and delivery module 110, at a moderate to high rate. Therefore, unlike the situation in the examples of FIGS. 27-30, there would be too much fluid (e.g., too constant) to yield a conductance vs. time graph with discrete peaks and troughs, for example as shown in FIG. 29 or FIG. 30. In order to accurately determine a sweat rate, an energy barrier can be used (e.g., as part of a flow regulator module 130) that induces periodic drop generation within wearable system 100. That is, the energy barrier can prevent further fluid flow until a critical volume of fluid builds up on one side of the barrier to cause a portion of the fluid to overcome the barrier. This process can occur periodically. It may be further assisted by an energetically favorable fluidic path being disposed after the energy barrier. For example, a lower energy waste module 140 (e.g., including a hydrophilic material, such as a wicking material or capillary pump) may be disposed downstream along a fluidic path of wearable system 100 from the energy barrier.

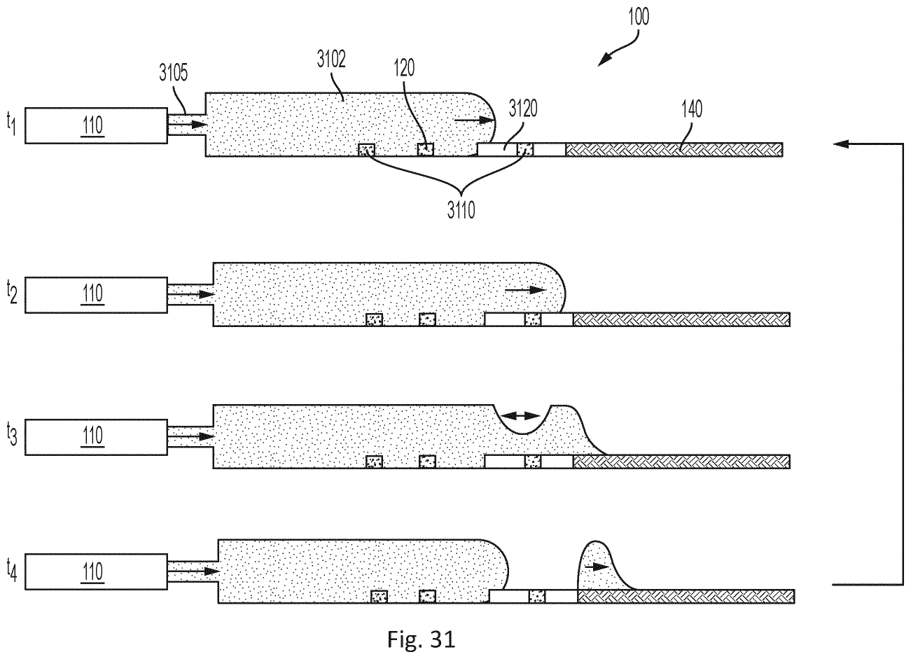

FIG. 31 is a schematic diagram of an example of periodic (e.g., pulsative) (e.g., regularly periodic) drop generation at the outlet of a fluidic capacitance across an energy barrier 3120 of a wearable system 100 that can be used with moderate to high sweat rates (S2). The fluid (e.g., sweat) 3102 is collected in fluid collection and delivery module 110, from where it flows into a fluidic capacitance (e.g., flowing through outlet 3105) and fills it (this is the initial condition of the system, which can take some initiation time) (t₁) thereby contacting main sensor module 120. Once the fluidic capacitance is filled, if more fluid 3102 is coming from fluid collection and delivery module 110 with a sufficient pressure, the fluid 3102 will tend to overcome the energy barrier 3120 (t₂), until it touches the energetically favorable path downstream (e.g., wicking material of waste module 140) (t₃). At this point, the fluid 3102 fragments into a droplet passing over the energy barrier 3120 (acting as a drop fragmenter) (t₄), which can be monitored by measuring the conductance between a sensing element (e.g., electrode) of a sweat presence monitoring device 3110 at (and in this case also on (e.g., embedded in)) the energy barrier 3120 and a sensing element (e.g., electrode) in the fluidic capacitance located upstream. Once the sweat drop has fragmented over the energy barrier 3120, the wearable system 100 returns to its state in t₁ and the process can repeat. The fluid collection and delivery module 110 may be a part of the fluidic capacitance.

The sweat presence monitoring device 3110 in this example is a conductance sensor shown here as comprising a pair of sensing elements that are electrodes. The electrodes may be electrically connected together (not explicitly shown) and operable to detect a conductive pathway through sweat when present between the electrodes from the one electrode to the other thereby outputting one or more signals; when the energy barrier 3120 is blocking the fluid 3102, then no pathway exists and there is no signal. One of the electrodes is at and on the energy barrier 3120 and one of the electrodes is in the fluidic capacitance (upstream of the energy barrier 3120 along a fluidic path of a wearable system 100). In some embodiments, a sensing element (e.g., electrode) is disposed after, and not at, an energy barrier (e.g., spaced by some distance). In some embodiments a sensing element (e.g., electrode) is disposed adjacent to, and not on, an energy barrier. Both electrodes can also be in the energy barrier, or one can be in the energy barrier and one downstream (in the energetically favorable environment downstream, e.g., waste module 140) along a fluidic path of the wearable system 100. At least one, but less than all, of the electrodes of the sweat presence monitoring device 3110, is disposed at or after the energy barrier 3120 and at least one, but less than all, of the electrodes of the sweat presence monitoring device 3110, is disposed before the energy barrier 3120.

Because one of the sensing elements of the sweat presence monitoring device 3110 is disposed at (e.g., on or adjacent to) the energy barrier 3120, or in some embodiments after, and the other is disposed before the energy barrier 3120, or in some embodiments at (e.g., on or adjacent to) the energy barrier 3120, relative to a fluidic path of a wearable system 100, the sweat presence monitoring device 3110 can detect periodic flow of fluid over the energy barrier 3120, thereby producing a conductance vs. time output with discrete curves (e.g., as shown in FIG. 29 or FIG. 30). Using the known geometry and energetic environment of wearable system 100, a sweat rate can be determined from the periodicity of the drop generation over the energy barrier 3120.

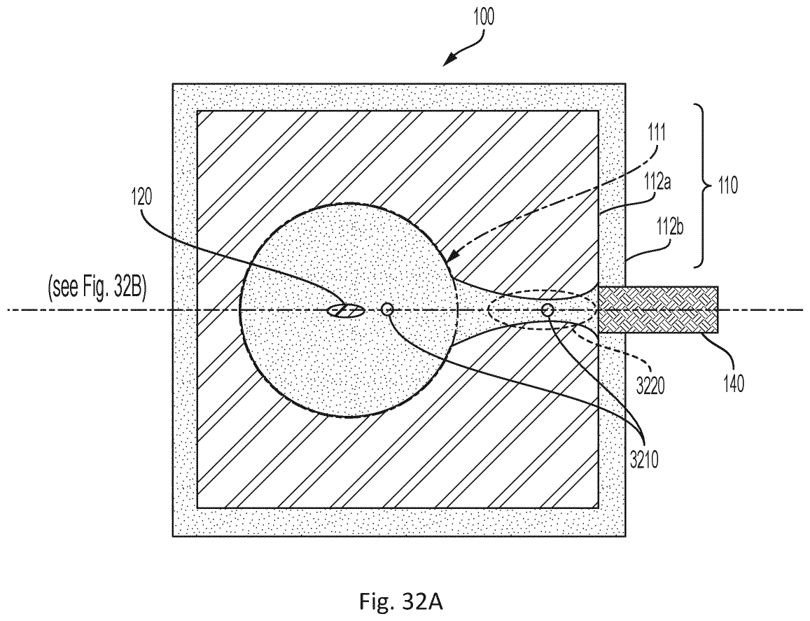

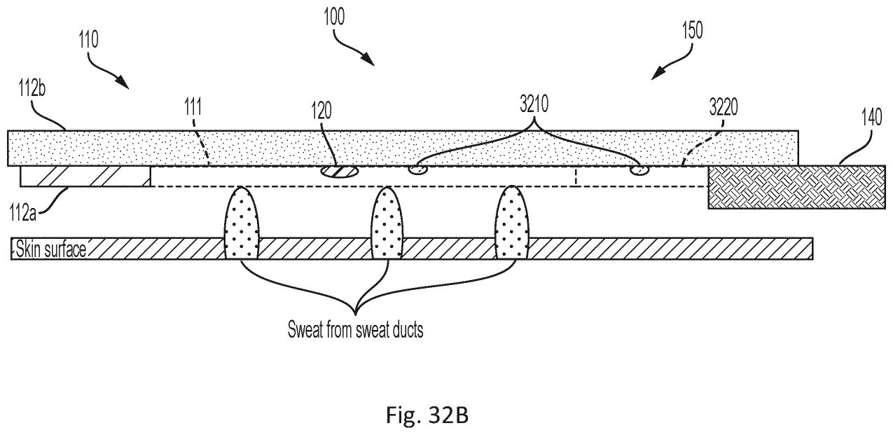

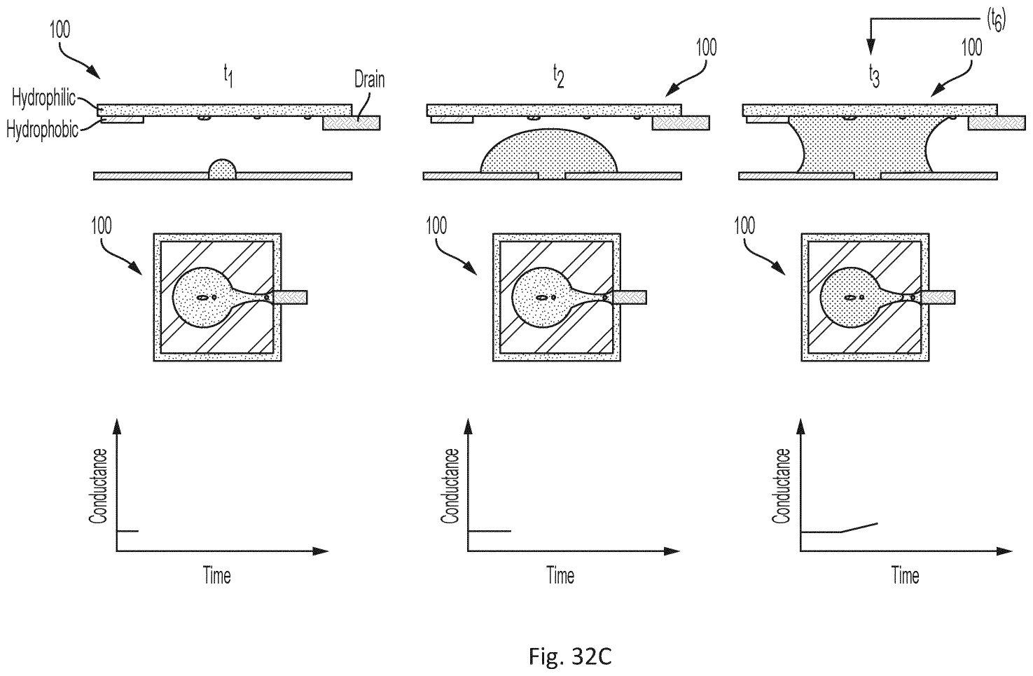

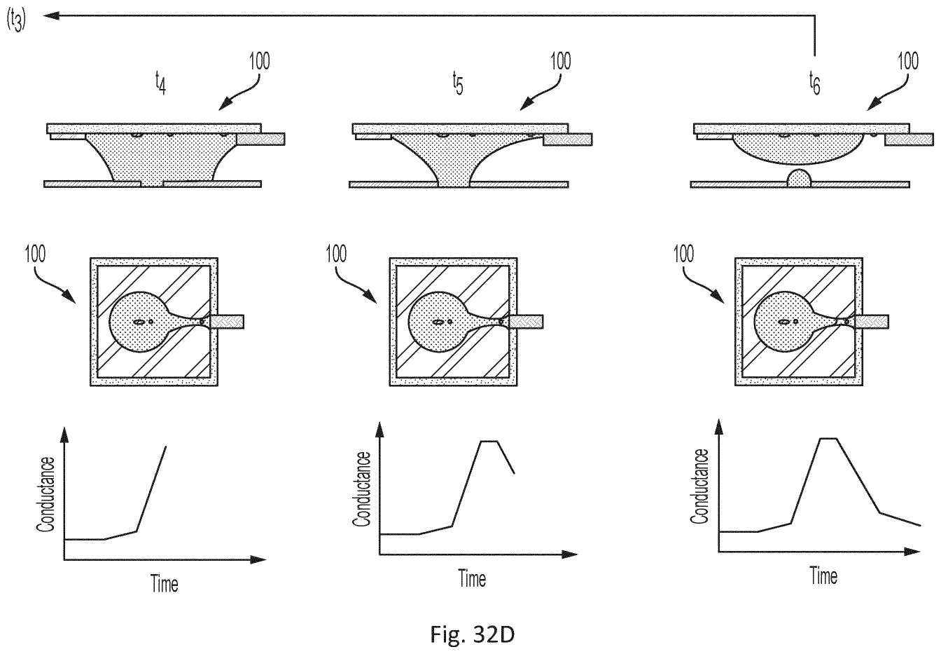

FIG. 32A is a top view of, and FIG. 32B is a cross-section view of, an example of a wearable system 100. The wearable system 100 includes a fluid collection and delivery module 110 that includes a fluidic collection zone 111 formed by a hydrophobic surface 112a comprising a confinement ring disposed on a hydrophilic surface 112b collectively acting as a fluidic capacitance, with a drop fragmenter structure as an energy barrier 3220 at the outlet. In this case, the energy barrier 3220, which is a drop fragmenter, is a constriction in the continuous hydrophobic surface 112a. In some embodiments a hydrophobic surface comprising a constriction is formed from two discrete hydrophobic portions (i.e., that are not connected together) (e.g., is a patterned hydrophobic surface). Waste module 140 is disposed downstream on a fluidic path of energy barrier 3220, which itself is downstream on the fluidic path of fluidic collection zone 111. The arrangement of the wearable system is such that fluid (e.g., sweat) will flow through the wearable system 100 in a manner similar to that illustrated in FIG. 31. Such a system, when put in close contact with the skin without touching (e.g., using a spacer layer, such as skin adhesive laminate) will tend to accumulate sweat within the hydrophobic confinement zone, thus forming the fluidic capacitance. A main sensor module 120 is disposed in the fluidic collection zone 111. The fluidic collection and delivery module 110 and the energy barrier 3220 form a fluidic capacitance.

The drop fragmenter structure (energy barrier 3220) is a structure consisting of a hydrophilic/hydrophobic pattern with a specific geometry, designed and dimensioned to promote the fragmentation of a droplet when the fluidic capacitance fills up. After fragmentation, the drop is wicked away by the hydrophilic drain of the waste module 140 (acting as an energetically favorable environment for the fluid (e.g., sweat)). The sweat presence monitoring device 3210 including two electrodes (one upstream in in the fluidic collection zone 111 and one downstream at the energy barrier 3220) that can be used to monitor fluid flow through the energy barrier 3220 to determine a sweat rate in a manner similar to as described with respect to FIG. 31.

FIGS. 32C and 32D are schematic diagrams of the sequence of events in during fluid (e.g., sweat) intake using the wearable system 100 shown in FIGS. 32A-B. The steps t₁ to t₃ show the initial filling of the fluidic collection zone 111 when sweat droplets start emerging on skin (only one droplet is shown, but there may be multiple droplets emerging from multiple sweat ducts addressed by the collection zone 111), then the steps t₃ to t₅ show the progression of the fluid across the energy barrier 3220 until it touches the energetically favorable environment for the fluid (the hydrophilic drain of waste module 140), and finally the step t₆ shows the fluid distribution in the wearable system 100 after the drop fragmentation. One or more spacer layers (e.g., skin adhesive) that maintain a minimal separation between hydrophilic surface 112b (acting as a flexible substrate for the wearable system 100) and the skin from which sweat is collected are not shown for clarity. After the step t₆ the sequence loops back to the step t₃, periodically. The corresponding conductance signals vs. time based on signal(s) output from the sweat presence monitoring device 3210 are also shown for each step. As can be seen, each drop fragmentation will result in an identifiable curve in the conductance vs. time plot for the sweat presence monitoring sensor, which can be used to determine sweat rate, for example based on a change in the intensity over time (e.g., periodicity of curves).

FIG. 33 is a cross-section view of a wearable system 100 that includes a fluid collection and delivery module 110 that includes a fluidic collection zone in fluid communication with a main sensor module 120 encapsulated in a closed fluidic channel 3331, and a hydrophobic step as an energy barrier 3320 at the outlet before waste module 140 that includes a hydrophilic drain (e.g., wicking material) at least a portion of which is disposed in the closed fluidic channel 3331 (or, in some embodiments, disposed at its outlet). The main sensor module 120 is disposed upstream along a fluidic path of the wearable system 100 of a sweat presence monitoring device 3310, of wetting sensor module 150, that includes two sensing elements that are electrodes. The downstream of the two electrodes is disposed at (and opposed within the closed fluidic channel 3331) the energy barrier 3320. The fluidic collection and delivery module 110, the closed fluidic channel 3331, and the energy barrier 3320 collectively form a fluidic capacitance.

The closed fluidic channel 3331 is defined by a flexible substrate 3330, a spacer/adhesive layer 3332, and a fluid channel lid layer 3334. Skin adhesive layer 3336 is disposed on an opposite side of the fluid channel lid layer 3334 from the closed fluidic channel 3331 and can be adhered to skin 3302. A cutout in the skin adhesive 3336, the fluid channel lid layer 3334, and the spacer/adhesive layer 3332 helps define the fluidic collection zone of the fluid collection and delivery module 110, which may, in some embodiments, be hydrophobic due to the materials used or further processed to have a hydrophobic surface (e.g., coated along the side wall(s)). This wearable system 100 is another implementation of the periodic fluid flow mechanism shown and described in FIG. 31. In this implementation, the fluidic capacitance is formed by the sweat collection zone of the fluid collection and delivery module 110 and the channel 3331 (which can be a 1D microchannel or a 2D slit channel). In some embodiments, the main sensor module 120 is disposed before an inlet to the closed microfluidic channel 3331. In some embodiments, the main sensor module 120 is disposed after a sensing element (e.g., electrode) of the sweat presence monitoring device 3310. The energy barrier 3320 is formed by a hydrophobic surface (in particular a hydrophobic step) on which an electrode of the sweat presence monitoring sensor 3310 (conductance sensor) (e.g., is inserted); another electrode is disposed after the main sensor module 120 and before the energy barrier 3320 along a fluidic path of the wearable system 100. The black arrows show the direction of fluid flow along the fluidic path. This wearable system 100 is another implementation of the periodic fluid flow mechanism shown and described in FIG. 31.

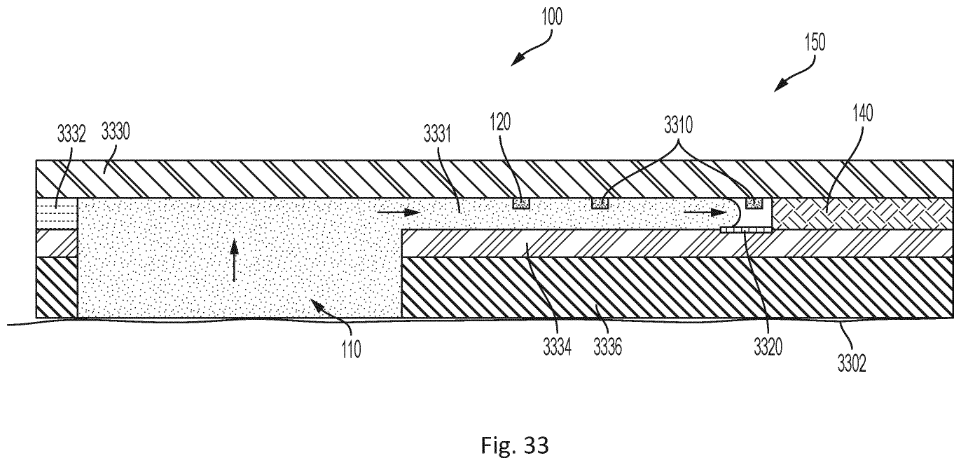

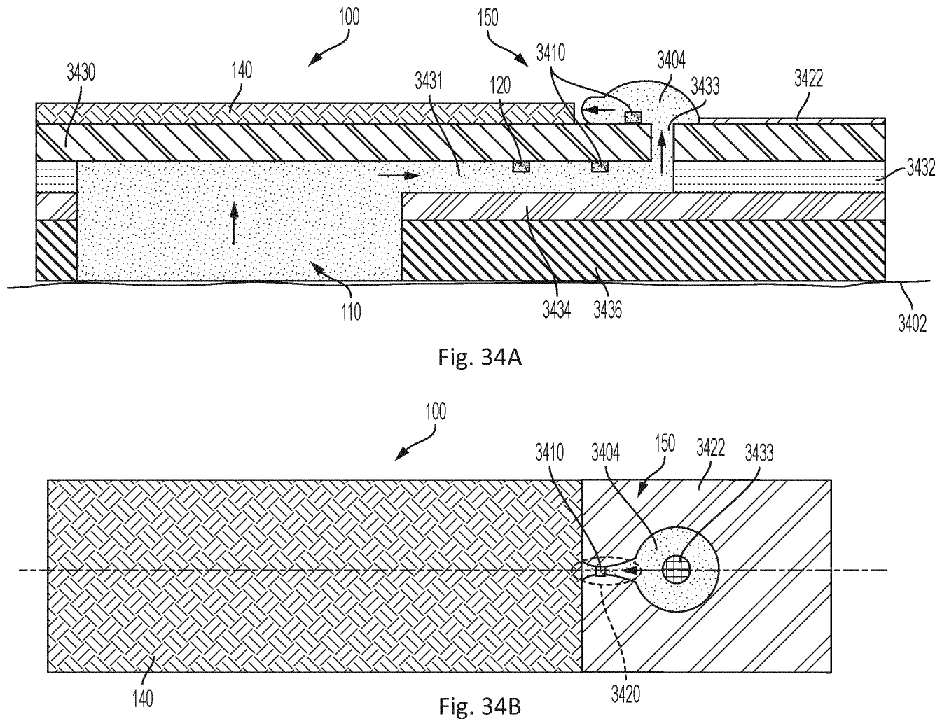

FIG. 34A is a top view of, and FIG. 34B is a cross-section view of, a wearable system 100 that includes a fluid collection and delivery module 110 that includes a fluidic collection zone in fluid communication with a main sensor module 120 encapsulated in a closed fluidic channel 3431 that flows into a fluid via 3433. Wetting sensor module 150 includes a hydrophobic ring (e.g., circular or polygonal) formed by hydrophobic surface 3422 also forms a constriction that acts as an energy barrier 3420 (a drop fragmenter in this case) at an outlet of the fluid via 3433 before a waste module 140 that includes a hydrophilic drain (e.g., wicking material). The hydrophobic surface 3422 forming the energy barrier 3420 and the waste module 140 are disposed on a common side of a flexible substrate 3430 opposite the closed fluidic channel 3431. As fluid flows through the wearable system 100, from the fluidic channel 3431 then through the via 3433, it will collect as a drop 3404 confined by the hydrophobic surface 3422 until there is sufficient energy (e.g., pressure) to overcome the energy barrier 3420 and continue flow energetically downhill to the waste module 140. The fluidic collection and delivery module 110, the closed fluidic channel 3431, the fluid via 3433, and the energy barrier 3420 collectively form a fluidic capacitance.

The main sensor module 120 is disposed upstream along a fluidic path of the wearable system 100 of a sweat presence monitoring device 3410, of wetting sensor module 150, that includes two sensing elements that are electrodes. The upstream of the two electrodes is disposed within the closed fluidic channel 3431. The downstream of the two electrodes is disposed along a fluidic path of the wearable system 100 at the energy barrier 3420 (in constriction of the hydrophobic surface 3422). The closed fluidic channel 3431 is defined by the flexible substrate 3430, a spacer/adhesive layer 3432, and a fluid channel lid layer 3434. The waste module 140 is disposed on a backside of the flexible substrate 3430. Such positioning of the waste module 140 can allow for dispersing fluid that has flowed through the wearable system 100 over a large area enabling rapid, diffuse removal (e.g., drying). Skin adhesive layer 3436 is disposed on an opposite side of the fluid channel lid layer 3434 from the closed fluidic channel 3431 and can be adhered to skin 3402. The fluid via 3433 is formed through the flexible substrate 3430. A cutout in the skin adhesive 3436, the fluid channel lid layer 3434, and the spacer/adhesive layer 3432 helps define the fluidic collection zone of the fluid collection and delivery module 110, which may, in some embodiments, be hydrophobic due to the materials used or further processed to have a hydrophobic surface (e.g., coated along the side wall(s)). This wearable system 100 is another implementation of the periodic fluid flow mechanism shown and described in FIG. 31. The black arrows show the direction of fluid flow along the fluidic path.

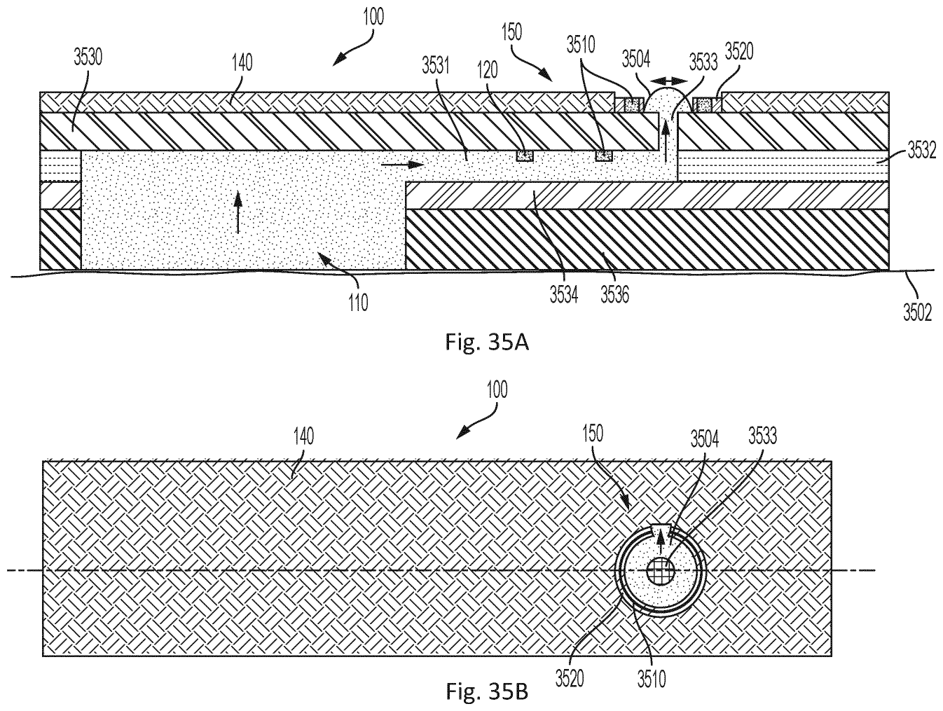

FIG. 35A is a top view of, and FIG. 35B is a cross-section view of, a wearable system 100 that includes a fluid collection and delivery module 110 that includes a fluidic collection zone in fluid communication with a main sensor module 120 encapsulated in a closed fluidic channel 3531 that flows into a fluid via 3533. Wetting sensor module 150 includes a hydrophobic ring (e.g., circular or polygonal) that acts as an energy barrier 3520 at an outlet of the fluid via 3533 disposed on a top surface of a substrate 3530 upstream of a waste module 140 that includes a hydrophilic drain (e.g., wicking material). Generally, the incorporation of a separate energy barrier ring (e.g., hydrophobic ring) is not required, for example if at least a portion of the whole top surface of the substrate 3530 is hydrophobic (e.g., by choice of material or through a hydrophobic coating). The waste module 140 is shown as being at least a portion of the top surface, but could be disposed elsewhere (for example if top surface, absent the waste module is disposed thereon or elsewhere). As fluid flows through the wearable system 100, from the fluidic channel 3531 then through the via 3533, it will collect as a drop 3504 confined by the hydrophobic ring forming the energy barrier 3520 until there is sufficient energy (e.g., pressure) to overcome the energy barrier 3520 and continue flow energetically downhill to the waste module 140. Energy barrier 3520 completely surrounds the outlet of fluid via 3533; as such, it is an isotropic energy barrier 3520. As fluid drop 3504 forms at the outlet, it will eventually be in a sufficiently high energetic state (e.g., have enough pressure caused by its size) to overcome the energy barrier 3520 and flow onto the hydrophilic drain of the waste module 140. Because the energy barrier 3520 is isotropic, local energetics (e.g., ambient conditions, gravity or others) will determine exactly where fluid overcomes the barrier 3520 (shown, arbitrarily, at the 12 o'clock position in FIG. 35B). Subsequent drops may fragment off of drop 3504 at different locations (e.g., due to a change in local conditions or a change in orientation of the wearable system 100). In some embodiments, an energy barrier only partially surrounds the outlet of a fluid via such that there is a preferential outlet for fluid after the outlet of the fluid via. The preferential outlet from the ring energy barrier may act as a drop fragmenter, for example, a functionally similar arrangement to FIGS. 34A-B can be formed where the hydrophobic surface is limited to a partial ring comprising a drop fragmenter. The fluidic collection and delivery module 110, the closed fluidic channel 3531, the fluid via 3533, and the energy barrier 3520 collectively form a fluidic capacitance.

The main sensor module 120 is disposed upstream along a fluidic path of the wearable system 100 of a sweat presence monitoring device 3510, of wetting sensor module 150, that includes two sensing elements that are electrodes. The upstream of the two electrodes is disposed within the closed fluidic channel 3531. The downstream of the two electrodes is disposed along a fluidic path of the wearable system 100 on the energy barrier 3520. The downstream electrode is a ring (e.g., circular or polygonal ring) that at least partially (and in this case completely) surrounds the outlet of the fluid via 3533. In some embodiments, the downstream electrode is disposed at and adjacent to (but not on) the energy barrier 3520, for example forming two concentric rings with the downstream electrode surrounding the energy barrier 3520. In some embodiments, where an energy barrier only partially surrounds an outlet of a fluid via thereby forming a drop fragmenter, an electrode is disposed in the open portion of the ring (in the drop fragmenter) or is itself a ring that only partially surrounds the outlet having an opening oriented with that of the energy barrier. The closed fluidic channel 3531 is defined by substrate 3530, a spacer/adhesive layer 3532, and a fluid channel lid layer 3534. The waste module 140 is disposed on a backside of the flexible substrate 3530. Skin adhesive layer 3536 is disposed on an opposite side of the fluid channel lid layer 3534 from the closed fluidic channel 3531 and can be adhered to skin 3502. The fluid via 3533 is formed through the flexible substrate 3530. A cutout in the skin adhesive 3536, the fluid channel lid layer 3534, and the spacer/adhesive layer 3532 helps define the fluidic collection zone of the fluid collection and delivery module 110, which may, in some embodiments, be hydrophobic due to the materials used or further processed to have a hydrophobic surface (e.g., coated along the side wall(s)). This wearable system 100 is another implementation of the periodic fluid flow mechanism shown and described in FIG. 31. The black arrows show the direction of fluid flow along the fluidic path.

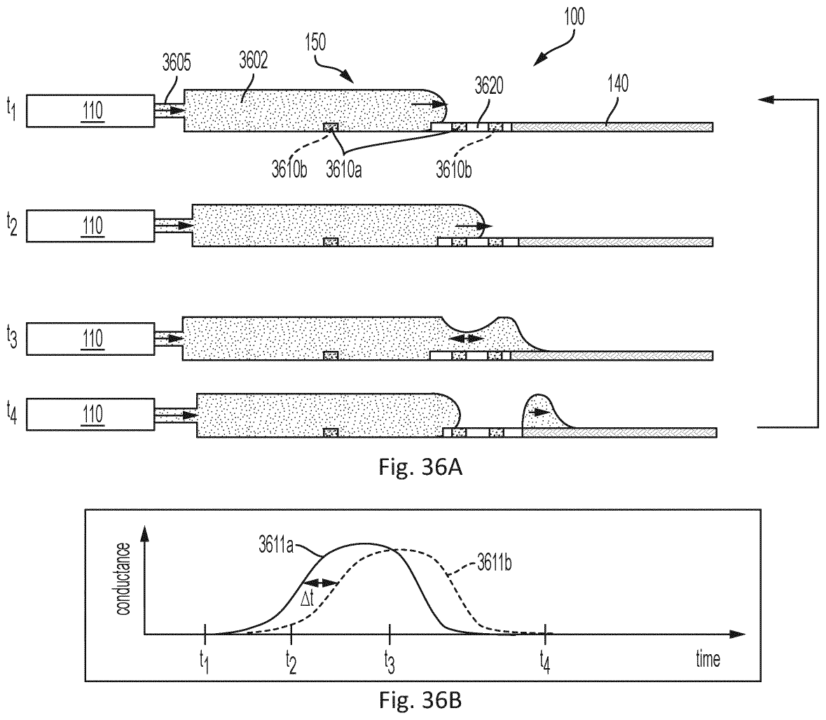

FIG. 36A is a schematic diagram of an example of periodic (e.g., pulsative) (e.g., regularly periodic) drop generation at the outlet of a fluidic capacitance across an energy barrier 3620 of a wearable system 100 that can be used with moderate to high sweat rates (S2). The fluid (e.g., sweat) 3602 is collected in fluid collection and delivery module 110, from where it flows into a fluidic capacitance (e.g., flowing through outlet 3605) and fills it (this is the initial condition of the system, which can take some initiation time) (t₁) thereby contacting main sensor module 120. Once the fluidic capacitance is filled, if more fluid 3602 is coming from fluid collection and delivery module 110 with a sufficient pressure, the fluid 3602 will tend to overcome the energy barrier 3620 (t₂), until it touches the energetically favorable path downstream (e.g., wicking material of waste module 140) (t₃). At this point, the fluid 3602 fragments into a droplet passing over the energy barrier 3620 (acting as a drop fragmenter) (t₄), which can be monitored by measuring the conductance between sensing elements (e.g., electrodes) of sweat presence monitoring devices 3610a-b. Once the sweat drop has fragmented over the energy barrier 3120, the wearable system 100 returns to its state in t₁ and the process can repeat. The fluid collection and delivery module 110 may contribute to the fluidic capacitance.

The sweat presence monitoring devices 3610a-b in this example are conductance sensor shown here as each comprising a pair of sensing elements that are electrodes. The electrodes may be electrically connected together (not explicitly shown) and operable to detect a conductive pathway through sweat when present between the electrodes from the one electrode to the other thereby outputting one or more signals; when the energy barrier 3620 is blocking the fluid 3602, then no pathway exists and there is no signal. For each of the sweat presence monitoring devices 3610a-b (conductance sensors 3610a-b), one of the electrodes is at and on the energy barrier 3620 and one of the electrodes is in the fluidic capacitance (upstream of the energy barrier 3620 along a fluidic path of a wearable system 100). In this example, one electrode (the upstream electrode) is shared between both of the sweat presence monitoring devices 3610a-b. If two or more electrodes in the energy barrier are used for conductance measurement versus an electrode in the confinement zone (as in the arrangement of conductance sensors 3610a-b in FIG. 36A), they will give a signal that is substantially the same when a drop travels across the energy barrier with a temporal phase shift corresponding to the delay of travel of the drops between the two or more electrodes (shown in FIG. 36B). Since the distance separating the electrodes is known by design, this temporal phase shift (between respective curves 3611a-b) can be used to compute the travel speed of the drop within the energy barrier 3620 (e.g., hydrophobic step), hence to monitor the dynamics of the drop fragmentation and speed. Sweat rate can be determined based on the temporal phase shift (e.g., and known geometry or other (e.g., energetic) properties of the fluidic environment). This approach can be extended to a plurality of sensing elements (e.g., electrodes) along the energy barrier 3620, for example three or more sensing elements.

The embodiments illustrated by FIGS. 27-30 are adapted for S1 conditions. If sweat rate is too high (in S2), then sweat is collected too continuously and the approach no longer functions since output from the sweat presence monitoring device(s) will be constant (or at least effectively constant). The embodiments illustrated by FIGS. 31-36B are adapted primarily for S2 conditions, but may be used in S1 conditions as well. While the illustrative embodiments described with respect to FIGS. 27 and 28 have been described as appropriate for use with low to moderate sweat rates (S1) and the illustrative embodiments described with respect to FIGS. 31-36B have been described as appropriate for use with moderate to high sweat rates (S2), it is contemplated, and will be readily understood, that, in some embodiments, a wearable system 100 includes a wetting sensor module 150 that includes structures (e.g., one or more sweat presence monitoring devices) that are, individually or collectively, suitable for use across a wide range of sweat rates. For example, a wearable system 100 may include a wetting sensor module 150 that includes a first sweat presence monitoring device including sensing elements disposed to measure sweat drops at low to moderate sweat rates (S1) where discrete sweat drops travel through the wearable system 100 individually and a second sweat presence monitoring device including sensing elements disposed to measure sweat flow at moderate to high sweat rates (S2), for example where sweat is pooled in a fluid collection zone (e.g., of a fluidic capacitance) and then flows through the rest of the wearable system 100. For example, in some embodiments a first sweat presence monitoring device includes sensing elements disposed exclusively before an energy barrier (along a fluidic path of the wearable system 100) and a second sweat presence monitoring device that includes sensing elements, at least one, and less than all, of which are disposed at or after the energy barrier (along the fluidic path) (e.g., as in a combination of FIG. 28 and one of FIGS. 31-36B), where the system is operable to use the first sweat presence monitoring device in S1 conditions and the second sweat presence monitoring device in S2 conditions.

In some embodiments, a sweat presence monitoring device may be arranged to enable sweat rate determination at high sweat rates (S2), for example with at least one sensing elements on each side of an energy barrier, while still being suitable for measuring low sweat rates (S1), for example by including other sensing element(s) or using a particular geometry or positioning. In some embodiments, a wetting sensor module 150 is disposed relative to a fluidic capacitance and an energy barrier such that even at low sweat rates (S1), a sweat rate can be determined, but after some delay while the fluidic capacitance slowly fills (where this delay can be appropriately accounted for, for example based on the known volume of the fluidic capacitance, in determining the sweat rate).

The embodiments illustrated in FIGS. 27-36B operate using passive fluid flow. That is, there is no powered component that drives the fluid flow (e.g., past an energy barrier and/or sweat presence monitoring device). For example, passive flow may be due to one or more of fluidic (e.g., hydraulic) capacitance, the inflow of sweat with sufficient pressure generated by the sweat ducts, and capillary action. In some embodiments, a wearable system 100 operates using active fluid flow, or a combination of active and passive fluid flow, where power is applied to drive fluid flow (e.g., past an energy barrier and/or sweat presence monitoring device), for example using a powered pump.

### Chemical Sensor Activation Module

The system may include a chemical sensor activation module 160 to chemically activate the sensor once (e.g., during fabrication), repeatedly (e.g., in order to reactivate the sensor during use) and/or on demand (for instance by actuation by a connected electronic device).

Chemical sensor activation module 160 may include all or some of the following component (possibly more than one instance of each): a fluidic inlet for the loading of the chemical solution, a fluidic reservoir to store it, a dedicated delivery fluidic channel or fluidic channel network (as described previously), and/or a dedicated flow regulation module (as described previously). It may be formed by a set of independent lines dedicated to a specific chemical or purpose (each line including for instance of inlet, reservoir, channel and flow regulation module).

The fluidic reservoir may include a cavity in which the chemical solution is loaded through the inlet once (e.g., at the time of fabrication) or repeatedly (e.g., by an electronic device connected to the system with fluidic connection, e.g., a user of the device). The fluidic reservoir may include an enlarged version of a fluidic channel (e.g., as described earlier) dimensioned (e.g., shaped and sized) in a way to store the required quantity of the chemical. For example, may be a closed fluidic channel that encloses (e.g., encapsulates) the collected fluid and prevent evaporations (e.g., as described earlier).

The chemical solution may contain at least one substance useful for the operation of at least one of the sensors of main sensor module 120. The chemical solution may be a solution providing necessary conditions for a sensor to perform well (or a buffer solution, e.g., a pH buffer), a substance used to functionalize a sensor, a substance used to reactivate or renew the functionalization of a sensor, a substance that reacts or forms a complex with the substance to be sensed, and/or a flushing or cleaning solution.

The dedicated flow regulation module(s) of chemical sensor activation module 160 may perform passively or actively (as described previously).

The dedicated delivery fluidic channel or channel network for chemical activation may intersect with the main delivery channel or channel network of the collection and delivery module. In certain embodiments, a reaction chamber is incorporated at the intersection of these modules.

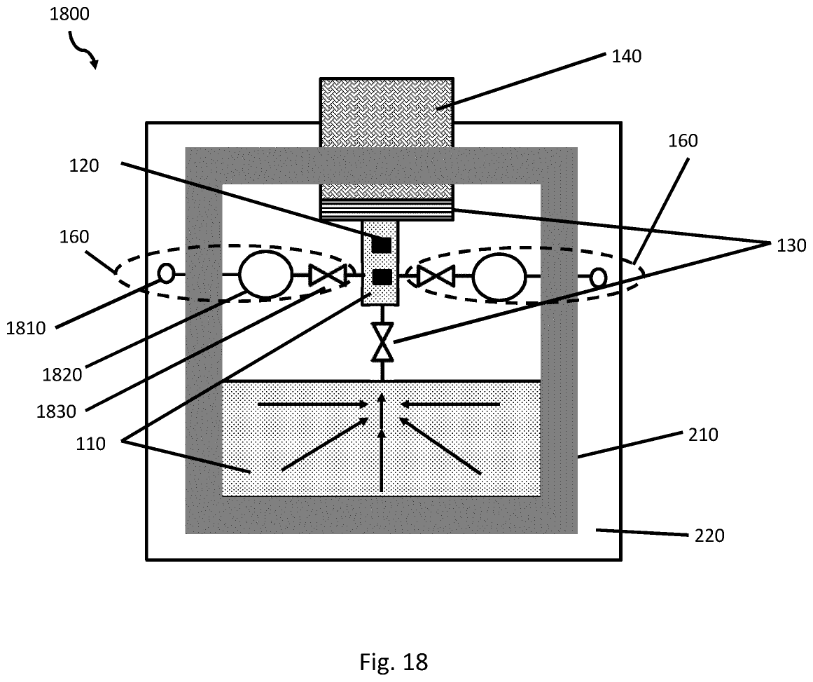

FIG. 18 shows an illustrative example of a system 1800 equipped with a chemical sensor activation module 160 with two lines and flow regulation based on a fluidic valve. Each line includes a separate set of inlet 1810, reservoir 1820, channel and fluidic valves 1830 and can be dedicated to a specific chemical or purpose.

Integration within a Microchip or Microchip Assembly

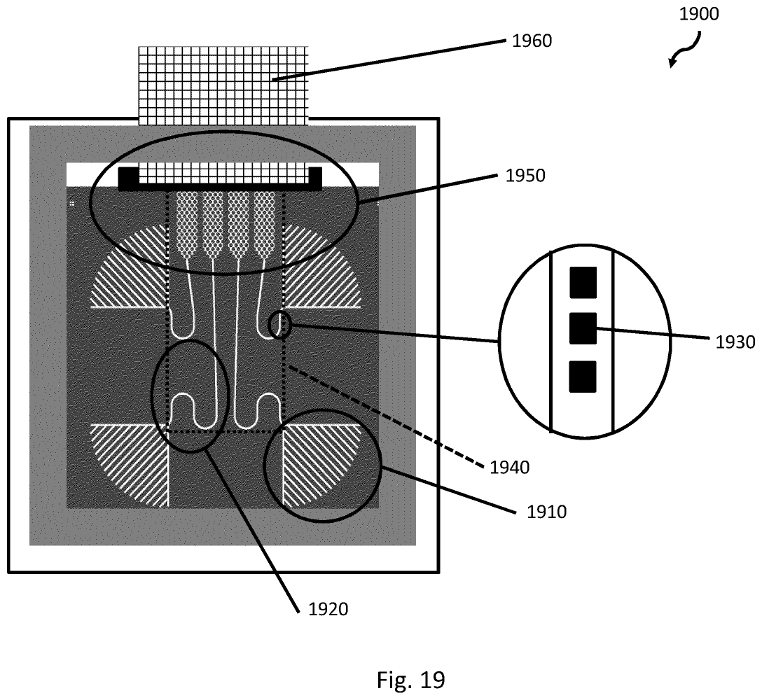

Some or part of the system (e.g., a portion (up to all) of the modules) described above may be integrated on a microchip or a microchip assembly. The microchip may further include a device to perform computation with memory to store calibration data. FIGS. 19 to 22 show illustrative examples of systems on a microchip.

FIG. 19 shows an illustrative example of a system 1900 that can be integrated on a microchip. System 1900 includes a collection and delivery module with four groups of arborescent collection structures 1910 located at four corners of the collection zone. The collection and delivery module can include microfluidic delivery channels 1920 which are disposed on top of or in the vicinity of sensors 1930, so that the fluid (e.g., sweat) collected can be transported to sensors 1930. An optional lid 1940 can locally close the channels if needed. System 1900 can also include a flow regulation module 1950 with a capillary pump and a hydrophobic barrier. A waste module 1960 with a wicking strip and an absorbent pad can be used to collect and dispose of the fluid. The hydrophobic barrier can be disposed between the channel outlet and the wicking strip.

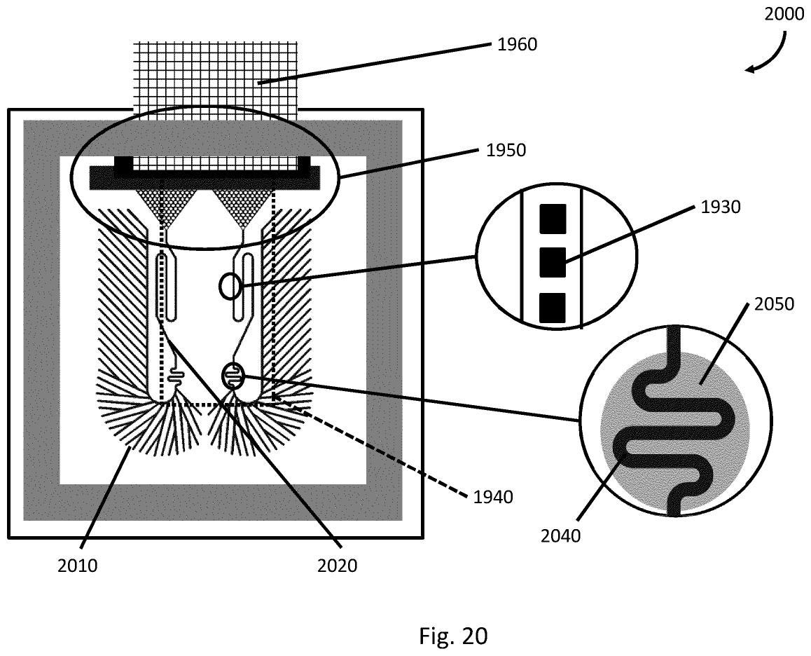

FIG. 20 shows another illustrative example of a system 2000 that can be integrated on a microchip. System 2000 includes two groups of arborescent collection structures 2010 to collect a fluid (e.g., sweat), and microfluidic delivery channels 2020 to transport the fluid to the sensor 1930. In certain embodiments, system 2000 includes serpentine structure 2040 to maximizing the overlap area of microfluidic delivery channel 2020 over a disk-shaped reference electrode 2050. An optional lid 1940 can locally close the channels if needed. System 2000 can also include a flow regulation module 1950 with a capillary pump and a hydrophobic barrier. A waste module 1960 with a wicking strip and an absorbent pad can be used to collect and dispose of the fluid.

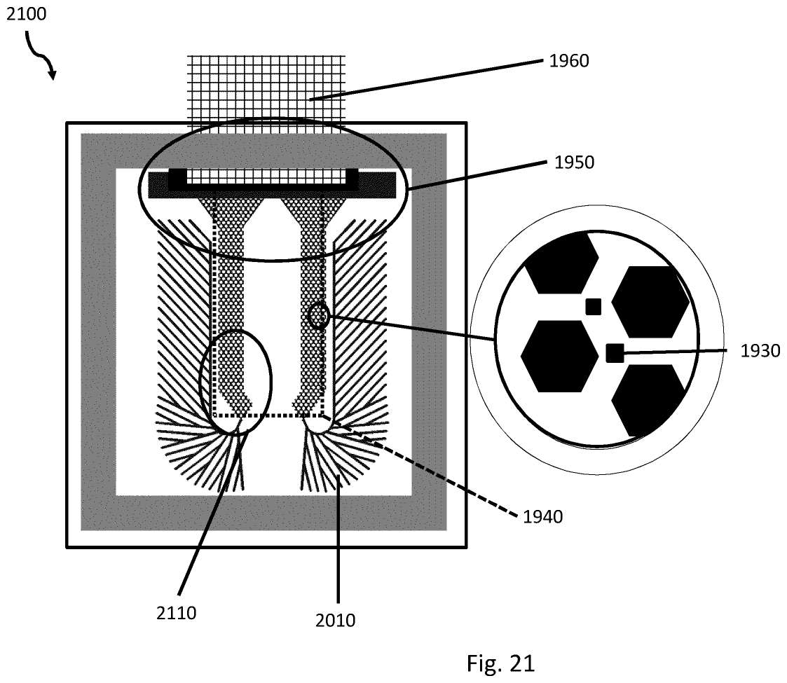

FIG. 21 shows another illustrative example of a system 2100 that can be integrated on a microchip. System 2100 includes two groups of arborescent collection structures 2010 to collect a fluid (e.g., sweat). System 2100 includes pillar-based delivery channels 2110 which transport the collected fluid (e.g., sweat) to the sensors 1930. An optional lid 1940 can locally close the channels if needed. System 2100 can also include a flow regulation module 1950 with a capillary pump and a hydrophobic barrier. A waste module 1960 with a wicking strip and an absorbent pad can be used to collect and dispose of the fluid.

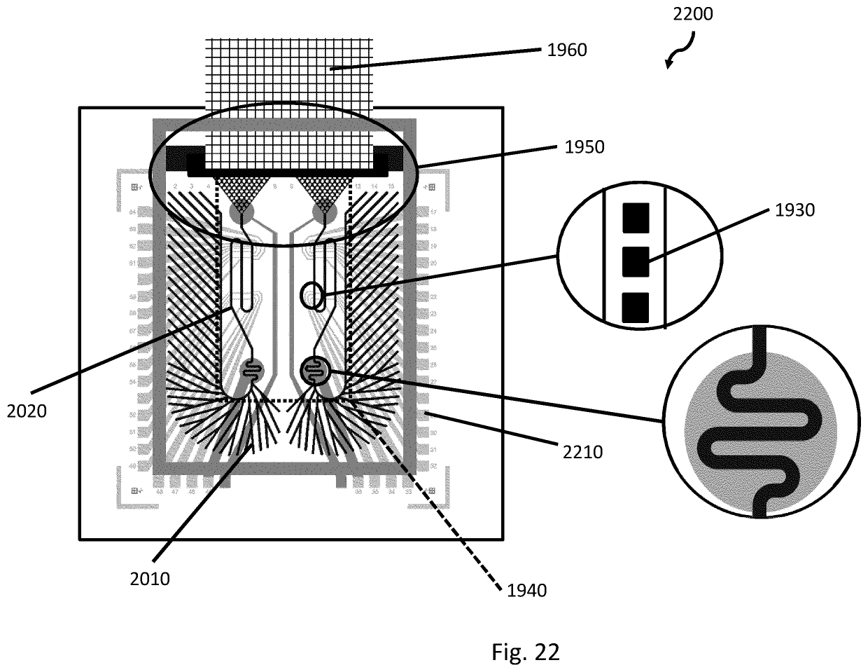

FIG. 22 shows another illustrative example of a system 2200 that can be integrated on a microchip. System 2200 includes a collection and delivery module, a flow regulation module and a waste module similar to system 2000 shown in FIG. 20, with two groups of arborescent collection structures 2010 and microfluidic delivery channel 2020. An optional lid 1940 can locally close the channels if needed. System 2200 can also include a flow regulation module 1950 with a capillary pump and a hydrophobic barrier. A waste module 1960 with a wicking strip and an absorbent pad can be used to collect and dispose of the fluid. System 2200 also includes a main sensor module similar to module 1400 shown in FIG. 14. In addition, system 2200 includes electrical connections 2210 to connect the sensors with an electronic circuit which can provide power to the sensors and collect measurement data from the sensors. For example, electrical connections 2210 can include pads connected to an electronic circuit via wire bonding, solder balls, etc.

The microchip or microchip assembly may be integrated on a printed-circuit board (PCB) e.g., a flex PCB). The PCB may be incorporated into a portable electronic device such as a patch, a wristband device, a watch, a smartphone, or a tablet computer.

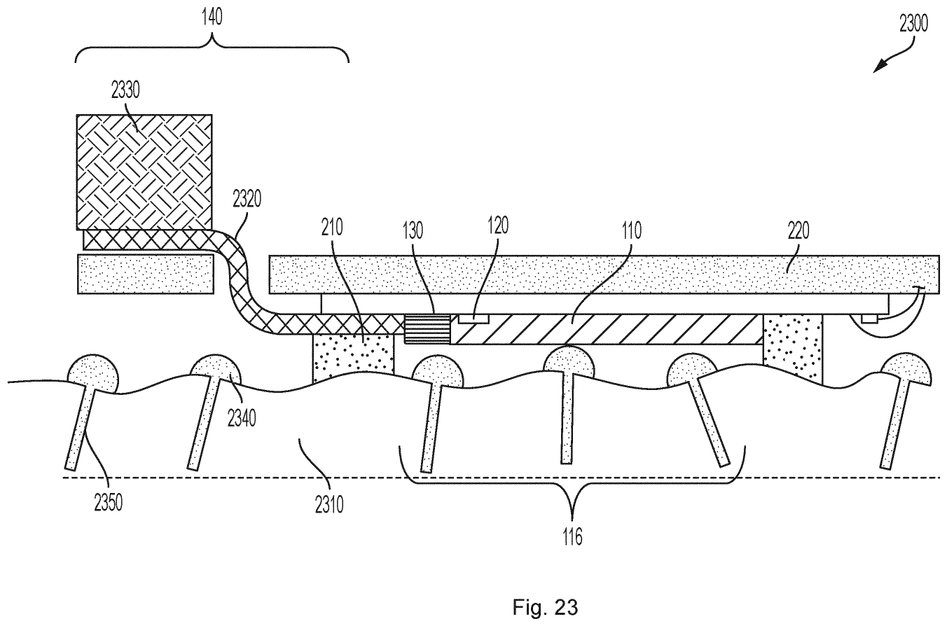

The system can be used for sweat collection and analysis when placed on human skin. FIG. 23 shows an illustrative example of a system 2300, embedded in a patch that is affixed to the human body 2310 with a skin adhesive. The system can be fabricated or disposed on a flexible substrate (e.g., a flex PCB). A collection zone 116 is defined by the surrounding sealant 210. In addition to collection and delivery module 110, main sensor module 120, flow regulation module 130, the system can include a waste module 140 with a wicking strip 2320 and an absorbent pad 2330. Sweat droplets 2340 coming from sweat ducts 2350 within the collection zone 116 can be collected by the collection and delivery module 110 and transported to the main sensor module 120 for detection and analysis.

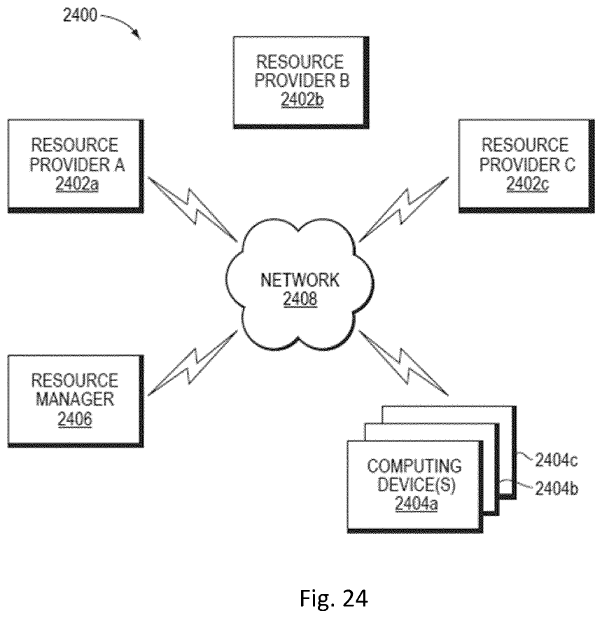

As shown in FIG. 24, an implementation of a network environment 2400 for use in the systems, methods, and architectures described herein, is shown and described. In brief overview, referring now to FIG. 24, a block diagram of an exemplary cloud computing environment 2400 is shown and described. The cloud computing environment 2400 may include one or more resource providers 2402a, 2402b, 2402c (collectively, 2402). Each resource provider 2402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 2402 may be connected to any other resource provider 2402 in the cloud computing environment 2400. In some implementations, the resource providers 2402 may be connected over a computer network 2408. Each resource provider 2402 may be connected to one or more computing device 2404a, 2404b, 2404c (collectively, 2404), over the computer network 2408.

The cloud computing environment 2400 may include a resource manager 2406. The resource manager 2406 may be connected to the resource providers 2402 and the computing devices 2404 over the computer network 2408. In some implementations, the resource manager 2406 may facilitate the provision of computing resources by one or more resource providers 2402 to one or more computing devices 2404. The resource manager 2406 may receive a request for a computing resource from a particular computing device 2404. The resource manager 2406 may identify one or more resource providers 2402 capable of providing the computing resource requested by the computing device 2404. The resource manager 2406 may select a resource provider 2402 to provide the computing resource. The resource manager 2406 may facilitate a connection between the resource provider 2402 and a particular computing device 2404. In some implementations, the resource manager 2406 may establish a connection between a particular resource provider 2402 and a particular computing device 2404. In some implementations, the resource manager 2406 may redirect a particular computing device 2404 to a particular resource provider 2402 with the requested computing resource.

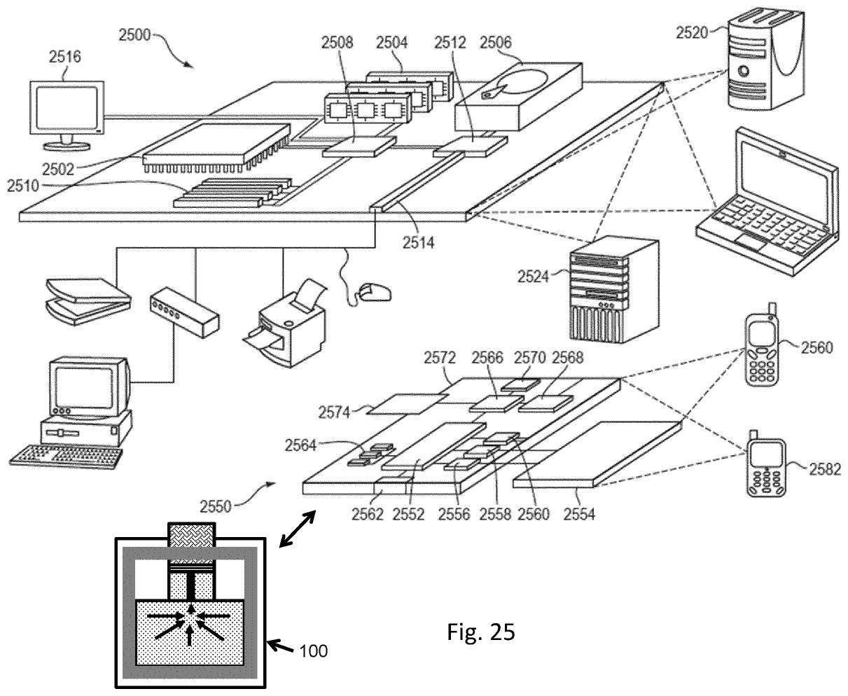

FIG. 25 shows an example of a computing device 2500 and a mobile computing device 2550 that can be used in the methods and systems described in this disclosure. For example, the computing device 2500 and/or mobile computing device 2550 can be in electronic communication with a system 100 for collecting and analyzing a fluid, as described. The computing device 2500 is intended to represent various forms of digital computers, such as laptops, desktops, workstations, personal digital assistants, servers, blade servers, mainframes, and other appropriate computers. The mobile computing device 2550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 2500 includes a processor 2502, a memory 2504, a storage device 2506, a high-speed interface 2508 connecting to the memory 2504 and multiple high-speed expansion ports 2510, and a low-speed interface 2512 connecting to a low-speed expansion port 2514 and the storage device 2506. Each of the processor 2502, the memory 2504, the storage device 2506, the high-speed interface 2508, the high-speed expansion ports 2510, and the low-speed interface 2512, are interconnected using various busses, and may be mounted on a common motherboard or in other manners as appropriate. The processor 2502 can process instructions for execution within the computing device 2500, including instructions stored in the memory 2504 or on the storage device 2506 to display graphical information for a GUI on an external input/output device, such as a display 2516 coupled to the high-speed interface 2508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 2504 stores information within the computing device 2500. In some implementations, the memory 2504 is a volatile memory unit or units. In some implementations, the memory 2504 is a non-volatile memory unit or units. The memory 2504 may also be another form of computer-readable medium, such as a magnetic or optical disk.

The storage device 2506 is capable of providing mass storage for the computing device 2500. In some implementations, the storage device 2506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 2502), perform one or more methods, such as those described above. The instructions can also be stored by one or more storage devices such as computer- or machine-readable mediums (for example, the memory 2504, the storage device 2506, or memory on the processor 2502).

The high-speed interface 2508 manages bandwidth-intensive operations for the computing device 2500, while the low-speed interface 2512 manages lower bandwidth-intensive operations. Such allocation of functions is an example only. In some implementations, the high-speed interface 2508 is coupled to the memory 2504, the display 2516 (e.g., through a graphics processor or accelerator), and to the high-speed expansion ports 2510, which may accept various expansion cards (not shown). In the implementation, the low-speed interface 2512 is coupled to the storage device 2506 and the low-speed expansion port 2514. The low-speed expansion port 2514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 2500 may be implemented in a number of different forms, as shown in the figure. For example, it may be implemented as a standard server 2520, or multiple times in a group of such servers. In addition, it may be implemented in a personal computer such as a laptop computer 2522. It may also be implemented as part of a rack server system 2524. Alternatively, components from the computing device 2500 may be combined with other components in a mobile device (not shown), such as a mobile computing device 2550. Each of such devices may contain one or more of the computing device 2500 and the mobile computing device 2550, and an entire system may be made up of multiple computing devices communicating with each other.

The mobile computing device 2550 includes a processor 2552, a memory 2564, an input/output device such as a display 2554, a communication interface 2566, and a transceiver 2568, among other components. The mobile computing device 2550 may also be provided with a storage device, such as a micro-drive or other device, to provide additional storage. Each of the processor 2552, the memory 2564, the display 2554, the communication interface 2566, and the transceiver 2568, are interconnected using various buses, and several of the components may be mounted on a common motherboard or in other manners as appropriate.

The processor 2552 can execute instructions within the mobile computing device 2550, including instructions stored in the memory 2564. The processor 2552 may be implemented as a chipset of chips that include separate and multiple analog and digital processors. The processor 2552 may provide, for example, for coordination of the other components of the mobile computing device 2550, such as control of user interfaces, applications run by the mobile computing device 2550, and wireless communication by the mobile computing device 2550.

The processor 2552 may communicate with a user through a control interface 2558 and a display interface 2556 coupled to the display 2554. The display 2554 may be, for example, a TFT (Thin-Film-Transistor Liquid Crystal Display) display or an OLED (Organic Light Emitting Diode) display, or other appropriate display technology. The display interface 2556 may comprise appropriate circuitry for driving the display 2554 to present graphical and other information to a user. The control interface 2558 may receive commands from a user and convert them for submission to the processor 2552. In addition, an external interface 2562 may provide communication with the processor 2552, so as to enable near area communication of the mobile computing device 2550 with other devices. The external interface 2562 may provide, for example, for wired communication in some implementations, or for wireless communication in other implementations, and multiple interfaces may also be used.

The memory 2564 stores information within the mobile computing device 2550. The memory 2564 can be implemented as one or more of a computer-readable medium or media, a volatile memory unit or units, or a non-volatile memory unit or units. An expansion memory 2574 may also be provided and connected to the mobile computing device 2550 through an expansion interface 2572, which may include, for example, a SIMM (Single In Line Memory Module) card interface or a DIMM (Double In Line Memory Module) card interface. The expansion memory 2574 may provide extra storage space for the mobile computing device 2550, or may also store applications or other information for the mobile computing device 2550. Specifically, the expansion memory 2574 may include instructions to carry out or supplement the processes described above, and may include secure information also. Thus, for example, the expansion memory 2574 may be provided as a security module for the mobile computing device 2550, and may be programmed with instructions that permit secure use of the mobile computing device 2550. In addition, secure applications may be provided via the DIMM cards, along with additional information, such as placing identifying information on the DIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 2552), perform one or more methods, such as those described above. The instructions can also be stored by one or more storage devices, such as one or more computer- or machine-readable mediums (for example, the memory 2564, the expansion memory 2574, or memory on the processor 2552). In some implementations, the instructions can be received in a propagated signal, for example, over the transceiver 2568 or the external interface 2562.

The mobile computing device 2550 may communicate wirelessly through the communication interface 2566, which may include digital signal processing circuitry where necessary. The communication interface 2566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 2568 using a radio-frequency. In addition, short-range communication may occur, such as using a Bluetooth®, Wi-Fi™, or other such transceiver (not shown). In addition, a GPS (Global Positioning System) receiver module 2570 may provide additional navigation- and location-related wireless data to the mobile computing device 2550, which may be used as appropriate by applications running on the mobile computing device 2550.

The mobile computing device 2550 may also communicate audibly using an audio codec 2560, which may receive spoken information from a user and convert it to usable digital information. The audio codec 2560 may likewise generate audible sound for a user, such as through a speaker, e.g., in a handset of the mobile computing device 2550. Such sound may include sound from voice telephone calls, may include recorded sound (e.g., voice messages, music files, etc.) and may also include sound generated by applications operating on the mobile computing device 2550.

The mobile computing device 2550 may be implemented in a number of different forms, as shown in the figure. For example, it may be implemented as a cellular telephone 2580. It may also be implemented as part of a smart-phone 2582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the modules (e.g. data aggregation module 2530, mapping module 2550, specifications module 2570) described herein can be separated, combined or incorporated into single or combined modules. The modules depicted in the figures are not intended to limit the systems described herein to the software architectures shown therein.

### Testing Environment Setup

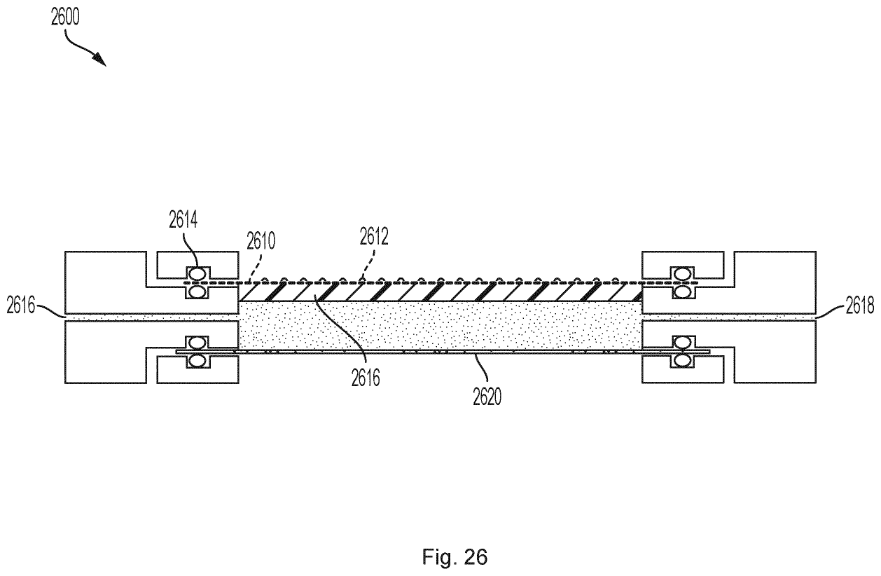

FIG. 26 is a schematic diagram of a device 2600 for mimicking the surface of skin (e.g., a wet surface). The performance of the biofluid collection and analysis system described herein can also be tested with this setup. The surface (e.g., a wet surface) may be a porous membrane 2610. The membrane may be a foil or a laminate, such as a polymer foil (e.g., PET, polycarbonate, polyimide, PDMS, or the like) or a metal foil (e.g., an aluminum, copper, or steel foil) that includes pores 2612. Pores 2612 can be patterned, for example, by cutting (e.g., laser-cutting), by machining (e.g., drilling or milling), by imprinting, etching (e.g., chemical etching, reactive-ion etching), and/or molding. Membrane 2610 may be mounted in a setup with O-ring 2614 such that a liquid is dispensed homogeneously (e.g., evenly) among a portion (up to all) of pores 2612. This can be achieved by inserting a material of higher fluidic resistance 2616 than pores 2612 on the backside of membrane 2610. This resistive material 2616 can be, for example, a hydrogel layer.

Still referring to FIG. 26, the surface (e.g., collection surface 118) may be a porous membrane 2610 designed to mimic sweating on skin, and therefore to reproduce some or all of the characteristics of human skin, such as pore (or sweat duct) density, pore (or sweat duct) size, hydrophobicity, roughness, patterning (e.g., the patterned “v-groove” network of skin), elasticity, the presence of a lipidic film, etc. A setup to fluidically connect such a membrane and mount it on a microscope may be used as a model to characterize, develop, and optimize the biofluid collection structures described herein. The performance of the biofluid collection and analysis system described herein can also be tested with this setup.

In certain embodiments, system 2600 includes an inlet 2616 for introducing a testing fluid into the system and an outlet 2618. System 2600 can also include a glass window 2620 for monitoring the flow distribution of the testing fluid within the system.

Elements of different implementations described herein may be combined to form other implementations not specifically set forth above. Elements may be left out of the processes, computer programs, databases, etc. described herein without adversely affecting their operation. In addition, the logic flows depicted in the figures do not necessarily require the particular order shown, or sequential order, to achieve desirable results. Various separate elements may be combined into one or more individual elements to perform the functions described herein. In view of the structure, functions and apparatus of the systems and methods described here, in some implementations.

The various described embodiments of the invention may be used in conjunction with one or more other embodiments unless technically incompatible.

While the disclosure has been particularly shown and described with reference to specific preferred embodiments, it should be understood by those skilled in the art that various changes in form and detail may be made therein without departing from the spirit and scope of the following claimed invention.

1. A system for automatically detecting a sweat rate of a subject, the system comprising:
a sweat collection and delivery module;
a wetting sensor module;
a processor; and
a memory having instructions thereon, the instructions, when executed by the processor, causing the processor to determine a sweat rate from one or more signals produced by the wetting sensor module in response to a presence of sweat from the sweat collection and delivery module.

2. The system of claim 1, comprising an energy barrier wherein the presence of sweat is a presence of sweat flowing past (e.g., over or through) the energy barrier.

3. The system of claim 1 or claim 2, comprising a drop fragmenter, wherein the presence of sweat is a presence of sweat being fragmented into drops by the drop fragmenter.

4. The system of claim 1, wherein the presence of sweat is a presence of discrete sweat drops.

5. The system of any one of the preceding claims, wherein the one or more signals produced by the wetting sensor module comprises an electrical signal.

6. The system of claim 5, wherein the electrical signal is a conductance (e.g., a conductance detected over time) (e.g., wherein the wetting sensor module comprises a conductance sensor, e.g., wherein the conductance sensor comprises one or more electrodes, e.g., wherein the one or more electrodes are actuated by an AC signal in a frequency range from about 1 kHz to about 100 kHz or wherein the one or more electrodes are actuated by a DC signal).

7. The system of claim 6, wherein the one or more signals comprises a plurality of channel conductance signals, and wherein the instructions cause the processor to determine the sweat rate from a detected frequency of conductance variation, based on the plurality of channel conductance signals.

8. The system of claim 6 or claim 7, wherein the one or more signals comprises a plurality of channel conductance signals, and wherein the instructions cause the processor to determine the sweat rate from a detected duty cycle, based on the plurality of channel conductance signals.

9. The system of any one of the preceding claims, wherein the one or more signals produced by the wetting sensor module comprises an optical signal (e.g., a fluorescence or other optical signal detected over time).

10. The system of any one of the preceding claims, comprising a functionalized sensor, wherein the instructions, when executed by the processor, determine a chemical and/or physical property of the sweat (e.g., a presence of, and/or concentration of, a biomarker) based on a signal from the functionalized sensor taking into account the detected sweat rate.

11. The system of any one of the preceding claims, further comprising a waste module for collecting, and optionally disposing, of sweat from the system after analysis.

12. The system of claim 11, wherein the waste module comprises a capillary pump and a waste reservoir.

13. The system of claim 11, wherein the waste module comprises a wicking material and a waste reservoir.

14. The system of claim 13, wherein the waste reservoir comprises an absorbent pad.

15. The system of any one of the preceding claims, further comprising a flow regulation module for regulating flow of sweat (e.g., sweat drops) into and/or through, and/or out of the system.

16. The system of any one of the preceding claims, wherein the system comprises a wearable housing (e.g., said housing non-invasively attachable and detachable from skin of the subject, e.g., via an adhesive surface).

17. The system of claim 16, wherein the wearable housing houses one or more members selected from the group consisting of the sweat collection and delivery module, the wetting sensor module, a power source (e.g., a battery), and the processor (e.g., and, optionally, the waste module and/or the flow regulation module).

18. The system of claim 16, wherein the processor is external to the wearable housing.

19. The system of any one of the preceding claims, comprising a microchip assembly for integrating at least two components selected from the group consisting of the collection and delivery module, the waste module, the flow regulation module, the processor, and the memory (e.g., wherein the microchip assembly comprises a printed circuit board).

20. The system of any one of the preceding claims, wherein the collection and delivery module comprises a surface with one or more collection structures (e.g., a fluid containment zone).

21. The system of claim 20, wherein the one or more collection structures comprise at least one fluidic channel or a fluidic channel network.

22. The system of claim 21, wherein the at least one fluidic channel or fluidic channel network comprises one or more members selected from the group consisting of a groove, an open or closed microfluidic channel, a two-dimensional channel defined by surface property contrast, and a channel made of a fixed gel matrix permeable to a fluid.

23. The system of any one of claims 20-22, wherein at least a portion of the at least one fluidic channel or fluidic channel network comprises pillar structures to facilitate fluid transport via capillary action.

24. The system of any one of claims 20-22, wherein the one or more collection structures comprise an arborescent channel network, and wherein the arborescent channel network comprises a plurality of branched channels.

25. A method for automatically detecting a sweat rate of a subject, the method comprising:
determining, by a processor of a computing device, a sweat rate from one or more signals produced by a wetting sensor module of a wearable (e.g., by a human subject) sweat rate detection system, the one or more signals produced in response to a presence of sweat in a sweat collection and delivery module of the system.

26. The method of claim 25, wherein the presence of sweat is a presence of sweat flowing past (e.g., over or through) an energy barrier.

27. The method of claim 25 or claim 26, wherein the presence of sweat is a presence of sweat being fragmented into drops by a drop fragmenter (e.g., that is the energy barrier).

28. The method of claim 25, wherein the presence of sweat is a presence of discrete sweat drops.

29. The method of claim 25, wherein the sweat rate detection system comprises the system of any one of claims 1 to 24.

30. A wearable system for determining a sweat rate of a subject, the system comprising:
a sweat presence monitoring device (e.g., conductance sensor) comprising a first sensing element and a second sensing element (e.g., first and second electrodes); and
an energy barrier,
wherein the first sensing element is disposed before the energy barrier along a fluidic path of the wearable system and the second sensing element is disposed at or after the energy barrier along the fluidic path.

31. The wearable system of claim 30, wherein the energy barrier comprises one or more of a hydrophobic surface (e.g., a hydrophobic surface comprising a constriction or a hydrophobic step), a surface tension barrier, a sterical obstacle, and a gravitational barrier.

32. The wearable system of claim 30 or claim 31, wherein the energy barrier is a drop fragmenter formed from a constriction in a hydrophobic surface.

33. The wearable system of any one of the preceding claims, wherein the second sensing element is disposed on or in the energy barrier.

34. The wearable system of claim 33, wherein the energy barrier is formed from a constriction in a hydrophobic surface and the second sensing element is disposed in the constriction.

35. The wearable system of any one of the preceding claims, wherein the second sensing element is a ring electrode disposed (i) on or (ii) adjacent to and at the energy barrier (e.g., and forming a circular or polygonal ring).

36. The wearable system of any one of the preceding claims, wherein the energy barrier is a ring (e.g., a circular or polygonal ring).

37. The wearable system of any one of the preceding claims, wherein the second sensing element is disposed in a closed fluidic channel.

38. The wearable system of any one of the preceding claims, wherein the energy barrier is disposed in a (e.g., the) closed fluidic channel.

39. The wearable system of any one claims 30-37, wherein the energy barrier and the second sensing element are disposed after a fluid via along the fluidic path.

40. The wearable system of claim 39, wherein one or more of the energy barrier and the second sensing element are disposed at an outlet of the fluid via (e.g., at least partially surround the outlet).

41. The wearable system of any one of the preceding claims, comprising a sweat collection and delivery module comprising an inlet disposed at a beginning of the fluidic path.

42. The wearable system of claim 41, wherein the sweat collection and delivery module comprises a fluidic collection zone comprising an opening at the inlet (e.g., a fluidic capacitance) (e.g., comprising a shaped hydrophobic surface disposed on a hydrophilic surface).

43. The wearable system of claim 41 or claim 42, wherein a wetting sensor module comprising the sweat presence monitoring device and the sweat collection and delivery module (e.g., and the main sensor module) are integrated and disposed on or in a common (e.g., flexible) substrate.

44. The wearable system of any one of the preceding claims, comprising a waste module (e.g., a capillary pump or hydrophilic wicking material) disposed along the fluidic path after (e.g., and adjacent to) the second sensing element (e.g., disposed at least partially in a closed fluidic channel).

45. The wearable system of any one of the preceding claims, comprising a second sweat presence monitoring device (e.g., conductance sensor) comprising the first sensing element and a third sensing element (e.g., the first and a third electrodes), wherein the third sensing element is disposed after the second sensing element along the fluidic path.

46. The wearable system of claim 45, wherein the sweat presence monitoring device and the second sweat presence monitoring device are together operable to output temporally phase shifted signals as sweat flows along the fluidic path.

47. The wearable system of any one of the preceding claims, wherein the first sensing element and the second sensing element are optical or electrical sensing elements.

48. The wearable system of claim 47, wherein the first sensing element and the second sensing element are electrodes that are disposed such that the sweat presence monitoring device is operable to output one or more signals when sweat is disposed continuously along the fluidic path from the first sensing element to the second sensing element.

49. The wearable system of any one of the preceding claims, comprising a main sensor module disposed before the first sensing element along the fluidic path.

50. The wearable system of any one of the preceding claims, comprising a wearable housing that houses the fluid collection and delivery module and a wetting sensor module comprising the sweat presence monitoring device, wherein the wearable housing is non-invasively attachable and detachable from skin of the subject.

51. The wearable system of claim 50, wherein the wearable housing comprises a skin adhesive, wherein the sweat collection and delivery module is disposed in fluid communication with skin of the subject when the skin adhesive is adhered to the skin.

52. The wearable system of any one of the preceding claims, comprising:
a processor; and
a memory having instructions thereon, wherein the instructions, when executed by the processor, cause the processor to determine a sweat rate from one or more signals produced by the sweat presence monitoring device in response to a presence of sweat.

53. The wearable system of claim 52, wherein:
a wetting sensor module comprises the sweat presence monitoring device and further comprises a second sweat presence monitoring device comprising the first sensing element and a third sensing element, the third sensing element being disposed at or after the energy barrier along the fluidic path, and
the instructions, when executed by the processor, cause the processor to determine the sweat rate based on a temporal phase shift between ones of the one or more signals and one or more signals produced by the second sweat presence monitoring device in response to a presence of sweat.

54. The wearable system of claim 52 or claim 53, wherein the instructions, when executed by the processor, cause the processor to determine the sweat rate based on a pulse frequency (e.g., duty cycle) of the signals.

55. The wearable system of claim 54, wherein the pulse frequency is due to individual discrete drops of sweat flowing along the fluidic path.

56. The wearable system of any one of the preceding claims, wherein the energy barrier is comprised in a flow regulation module.

57. A method of determining a sweat rate of a subject, the method comprising:
collecting sweat from skin of the subject in a wearable system comprising one or more sweat presence monitoring devices (e.g., conductance sensor(s));
flowing the sweat over the one or more sweat presence monitoring devices over a period of time;
detecting that the sweat is present with the one or more sweat presence monitoring devices thereby causing the one or more sweat presence monitoring devices to output one or more signals during the period of time; and
automatically determining, by a processor (e.g., integrated in the wearable system), a sweat rate based on a change in the one or more signals over the period of time.

58. The method of claim 57, wherein flowing the sweat comprises periodically flowing (e.g., at regular periods) portions of the sweat past (e.g., over or through) an energy barrier and detecting that the sweat is present occurs only as each of the portions flows past the energy barrier (e.g., wherein the energy barrier comprises one or more of a hydrophobic surface, a surface tension barrier, a sterical obstacle, and a gravitational barrier).

59. The method of claim 57 or claim 58, wherein flowing the sweat comprises fragmenting discrete drops from the sweat by flowing the sweat past (e.g., over or through) a drop fragmenter and detecting that the sweat is present occurs only as each of the discrete drops is fragmented.

60. The method of claim 57, wherein the sweat comprises discrete drops of sweat and flowing the sweat comprises individually flowing the discrete drops over the one or more sweat presence monitoring devices.

61. The method of claim 60, wherein flowing the sweat comprises fragmenting discrete drops from the sweat by flowing the sweat past (e.g., over or through) a drop fragmenter and detecting that the sweat is present occurs only as each of the discrete drops is fragmented.

62. The method of any one of claims 57-61, comprising detecting that the sweat is present with the one or more sweat presence monitoring devices after a portion of the sweat has exited a fluid via.

63. The method of claim 62, wherein a portion of at least one of the one or more sweat presence monitoring devices (e.g., an electrode or optical sensor of the sweat presence monitoring device) (e.g., and the energy barrier) (e.g., and the drop fragmenter) is (e.g., are) disposed after the fluid via along a fluidic path of the sweat through the wearable system (e.g., and at least partially around the fluid via).

64. The method of any one of claims 57-63, wherein collecting the sweat from the skin comprises collecting the sweat in a fluidic collection zone (e.g., comprising a shaped hydrophobic surface disposed on a hydrophilic surface).

65. The method of any one of claims 57-64, wherein each of the one or more sweat presence monitoring devices comprises a first electrode and a second electrode and detecting that the sweat is present comprises measuring a conductance through the sweat from the first electrode to the second electrode (e.g., and wherein the second electrode is disposed at or after an energy barrier) (e.g., and wherein the second electrode is disposed at or after a drop fragmenter).

66. The method of any one of claims 57-64, wherein each of the one or more sweat presence monitoring devices is an optical sweat presence monitoring device comprising one or more optical sensing elements (e.g., that sense(s) fluorescence).

67. The method of any one of claims 57-66, wherein the change is a change in intensity [e.g., determined using frequency spectrum analysis (e.g. fast Fourier transform, spectrogram or FFT based time-frequency analysis) or a period measuring technique (e.g. rising/fall edge detection)].

68. The method of any one of claims 57-67, wherein the one or more signals is a plurality of signals from a plurality of sweat presence monitoring devices and the change is a temporal phase shift between ones of the plurality of signals from different ones of the plurality of sweat presence monitoring devices.

69. The method of any one of claims 57-68, wherein the one or more signals are distributed over time to form a pulse-width-modulated composite signal (e.g., with a constant duty cycle) and automatically determining the sweat rate is based on the pulse-width-modulated composite signal.

70. The method of any one of claims 57-69, wherein the sweat rate is a total or average liquid generation rate.

71. The method of any one claims 57-70, wherein:
the wearable system comprises the processor and a memory having instructions stored thereon,
outputting the one or more signals comprises receiving the one or more signals in the processor, and
the sweat rate is automatically determined by the processor (i) during or after the period of time upon receiving at least one of the one or more signals and (ii) using the instructions.

72. The method of any one of claims 57-71, wherein flowing the sweat over the one or more sweat presence monitoring devices comprises passively flowing the sweat (e.g., due to one or more of hydraulic capacitance and capillary action).

73. The method of any one of claims 57-71, wherein flowing the sweat over the one or more sweat presence monitoring devices comprises actively flowing the sweat (e.g., by a powered pump).

74. A wearable system for determining a sweat rate of a subject, the system comprising:
a wetting sensor module comprising two sweat presence monitoring devices each comprising sensing elements (e.g., electrodes or optical sensing elements); and
wherein the two sweat presence monitoring devices are disposed along a fluidic path of the wearable system such that the two sweat presence monitoring devices are operable to output temporally phase shifted signals (e.g., electrical or optical signals) corresponding to sweat flow along the fluidic path.

75. The wearable system of claim 74, wherein the two sweat presence monitoring devices comprise a common sensing element.

76. The wearable system of claim 74 or claim 75, comprising a fluid collection and delivery module, wherein an inlet of the fluid collection and delivery module (e.g., a fluid containment zone) is disposed at a beginning of the fluidic path.

77. The wearable system of any one of claims 74-76, comprising a processor and a memory having instructions thereon, wherein the instructions, when executed by the processor, cause the processor to automatically determine a sweat rate from the temporally phase shifted signals.

78. The wearable system of any one of claims 74-77, wherein the system is one according to any one of claims 1-24 and 30-56.
